(19) United States
(12) Patent Application Publication
(10) Pub. No.: US 2017/0267667 A1
(43) Pub. Date: Sep. 21, 2017

(54) ALKENYLDIARYLMETHANES AS NON-NUCLEOSIDE REVERSE TRANSCRIPTASE INHIBITORS FOR ANTI-HIV-1 CHEMOTHERAPY

(71) Applicants: Purdue Research Foundation, West Lafayette, IN (US); Josai University, Sakado-shi (JP)

(72) Inventors: Mark S. Cushman, West Lafayette, IN (US); Takeshi Sakamoto, Hiki Gun (JP)

(73) Assignee: Purdue Research Foundation, West Lafayette, IN (US)

(21) Appl. No.: 15/459,041

(22) Filed: Mar. 15, 2017

Related U.S. Application Data
(60) Provisional application No. 62/308,249, filed on Mar. 15, 2016.

(51) Int. Cl. C07D 413/10; A61K 31/423; A61K 31/421; C07D 271/10; A61K 31/265; C07D 263/24; C07D 263/58; C07D 261/20; C07C 327/02; A61K 31/4245; C07D 271/06

(52) U.S. Cl. CPC C07D 413/10; A61K 31/4245; A61K 31/423; A61K 31/421; C07D 271/10; C07D 271/06; C07D 263/24; C07D 263/58; C07D 261/20; C07C 327/02; A61K 31/265

(57) ABSTRACT

The invention disclosed herein pertains to compounds of alkenyldiarylmethanes (ADAMs) as non-nucleoside reverse transcriptase inhibitors (NNRTIs) for the treatment of patients with acquired-immune deficiency syndrome (AIDS). Also described are methods for treating AIDS patients using the described alkenyldiarylmethane compounds.

### CROSS-REFERENCE TO RELATED APPLICATIONS

The present U.S. patent application is related to and claims the priority benefit of the U.S. Provisional Patent Application Ser. No. 62/308,249, filed Mar. 15, 2016, the contents of which are hereby incorporated by reference in their entirety into the present disclosure.

### BACKGROUND

This section introduces aspects that may help facilitate a better understanding of the disclosure. Accordingly, these statements are to be read in this light and are not to be understood as admissions about what is or is not prior art.

Human immunodeficiency virus type 1 (HIV-1) targets CD4-positive T lymphocytes and causes acquired immune deficiency syndrome (AIDS). According to a Joint United Nations Programme on HIV and AIDS (UNAIDS) report, AIDS-related deaths fell to 1.5 million people in 2013 and the number of people living with HIV also fell. However, worldwide, 2.1 million people were infected with HIV. In the antiretroviral therapy (ART) started in the mid-1990s, combinations of anti-HIV-1 agents with different mechanisms of action were used in the treatment of HIV infection, resulting in a decrease in the rate of AIDS progression. Anti-HIV-1 agents may be viewed as falling into the following categories: nucleoside reverse transcriptase inhibitors (NRTIs), non-nucleoside reverse transcriptase inhibitors (NNRTIs), protease inhibitors (PIs), integrase inhibitors (INIs), fusion inhibitors (FIs), and C—C chemokine receptor inhibitors (CCR₅Is). Although combinations of these agents are effective in the treatment of AIDS, the necessity for long-term administration of anti-AIDS drugs results in problems such as a decline in adherence, the development of drug resistance, and long-term side effects (Stevenson, M., Sci. Am. 2008, 299, 78-83). There are still unmet needs in dealing with HIV-1 due to its high mutation rate, which has resulted in continuous demand for better and more efficient treatment.

Previously we have reported alkenyldiarylmethane (ADAM) derivatives as NNRTIs (Sakamoto, T., et al., J. Med. Chem. 2007, 50, 3314-3321; Cullen, M. D., et al., J. Med. Chem. 2007, 50, 4854-4867). Those ADAMs were synergistic with the NRTI AZT and displayed enhanced activity when tested against AZT-resistant strains of HIV-1. However, those ADAMs bear three methyl esters that may be hydrolyzed by nonspecific esterase present in blood plasma, which leads to an inactive metabolite. Further improvements in those original compounds are needed.

### BRIEF SUMMARY OF THE INVENTION

In some illustrative embodiments, this invention is related to a compound of formula (I)

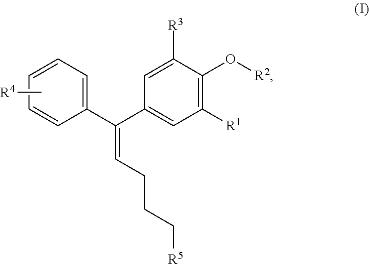

or a pharmaceutically acceptable salt, hydrate, prodrug, polymorph, or solvate thereof, wherein
R¹ is hydrogen, an alkyl, alkenyl, alkynyl, heteroalkyl, or heteroalkenyl;
R² is an alkyl or acyl;
R³ is an acyl or an imidoyl;
R⁴ represents five substituents each independently selected from the group consisting of hydrogen, halo, azido, cyano, nitro, hydroxy, amino, thio, acyl, acyloxy, alkyl, alkenyl, heteroalkyl, heteroalkenyl, heterocyclyl, and aryl, each of which is optionally substituted; or any two adjacent substituents that are taken together with the attached carbons to form an optionally substituted heterocycle and each of other three substitutents is defined as above; and
R⁵ is acyl, alkyl, alkenyl, heteroalkyl, heteroalkenyl, heterocyclyl, cycloalkyl, cycloalkenyl, cycloheteroalkyl, cycloheteroalkenyl, aryl, arylalkyl, or arylalkenyl, each of which is optionally substituted.

In some preferred embodiments, the compound of this invention is represented by formula (I) wherein R¹ is a C₁-C₆ alkyl.

In some preferred embodiments, the compound of this invention is represented by formula (I) wherein R² is a C₁-C₆ alkyl.

In some preferred embodiments, the compound of this invention is represented by formula (I) wherein R³ is an N-alkoxyimidoyl halide.

In some preferred embodiments, the compound of this invention is represented by formula (I) wherein R³ is an acyl thioester and R⁵ is an N-alkoxyimidoyl halide.

In some preferred embodiments, the compound of this invention is represented by formula (I) wherein R⁴ represents five substituents wherein one of the five substituents is a cyano and other four are hydrogen.

In some preferred embodiments, the compound of this invention is represented by formula (I) wherein R⁴ represents five substituents wherein two adjacent substituents are taken together with the attached carbons/heteroatoms to form an optionally substituted cyclic or heterocyclic moiety.

In some preferred embodiments, the compound of this invention is represented by formula (I) wherein R⁵ is an N-alkoxyimidoyl halide.

In some preferred embodiments, the compound of this invention includes

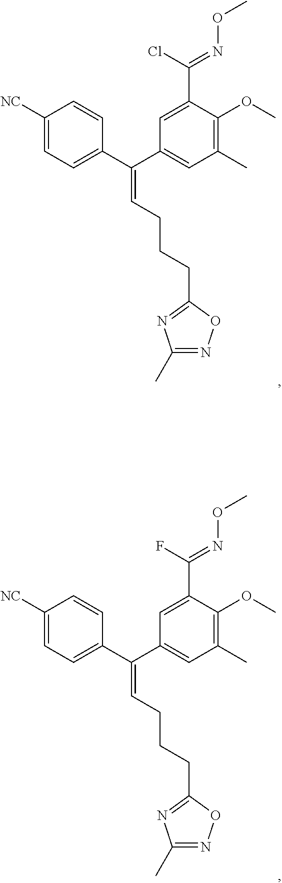 [SMILES: CO/N=C(\Cl)c1cc(/C(=C\CCCc2nc(C)no2)c2ccc(C#N)cc2)cc(C)c1OC ; CO/N=C(\F)c1cc(/C(=C\CCCc2nc(C)no2)c2ccc(C#N)cc2)cc(C)c1OC]

or a pharmaceutically acceptable salt, hydrate, prodrug, polymorph, or solvate thereof.

It is to be understood that all possible combinations of the various genera and subgenera of each of R¹, R², R³, R⁴, and R⁵ in formula (I) described herein represent additional illustrative embodiments of compounds of the invention described herein. It is also to be understood that each of those additional illustrative embodiments of compounds may be used in any of the compositions, methods, and/or uses described herein.

In some embodiments, this invention is related to a pharmaceutical composition comprising a compound represented by formula (I), or a pharmaceutically acceptable salt thereof, together with one or more diluents, excipients or carriers.

In some embodiments, this invention is related to a pharmaceutical composition comprising a compound represented by formula (I), or a pharmaceutically acceptable salt thereof, in combination with one or more other therapeutically active compounds by the same or different modes of action, together with one or more diluents, excipients or carriers.

In some embodiments, this invention is related to a method for treatment of a patient with HIV infection comprising administration of a therapeutically effective amount of a compound represented by formula (I), or a pharmaceutically acceptable salt thereof, together with one or more diluents, excipients or carriers, to the patient in need of relief from said infection.

In some embodiments, this invention is related to a method for treatment of a patient with HIV infection comprising administration of a therapeutically effective amount of a compound represented by formula (I), or a pharmaceutically acceptable salt thereof, in combination with one or more other therapeutically active compounds by the same or different modes of action, together with one or more diluents, excipients or carriers, to the patient in need of relief from said infection.

These and other features, aspects and advantages of the present invention will become better understood with reference to the following description and claims.

### DETAILED DESCRIPTION OF THE INVENTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

The term “substituted” as used herein refers to a functional group in which one or more hydrogen atoms contained therein are replaced by one or more non-hydrogen atoms. The term “functional group” or “substituent” as used herein refers to a group that can be or is substituted onto a molecule. Examples of substituents or functional groups include, but are not limited to, a halogen (e.g., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

The term “alkyl” as used herein refers to substituted or unsubstituted straight chain and branched alkyl groups and cycloalkyl groups having from 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to substituted or unsubstituted straight chain and branched divalent alkenyl and cycloalkenyl groups having from 2 to 20 carbon atoms(C₂-C₂₀), 2 to 12 carbons (C₂-C₁₂), 2 to 8 carbon atoms (C₂-C₈) or, in some embodiments, from 2 to 4 carbon atoms (C₂-C₄) and at least one carbon-carbon double bond. Examples of straight chain alkenyl groups include those with from 2 to 8 carbon atoms such as —CH═CH—, —CH═CHCH₂—, and the like. Examples of branched alkenyl groups include, but are not limited to, —CH═C(CH₃)— and the like.

An alkynyl group is the fragment, containing an open point of attachment on a carbon atom that would form if a hydrogen atom bonded to a triply bonded carbon is removed from the molecule of an alkyne. The term “hydroxyalkyl” as used herein refers to alkyl groups as defined herein substituted with at least one hydroxyl (—OH) group.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “aryl” as used herein refers to substituted or unsubstituted cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The term “aralkyl” and “arylalkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” as used herein refers to substituted or unsubstituted aromatic and non-aromatic ring compounds containing 3 or more ring members, of which, one or more is a heteroatom such as, but not limited to, B, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈).

A heteroaryl ring is an embodiment of a heterocyclyl group. The phrase “heterocyclyl group” includes fused ring species including those that include fused aromatic and non-aromatic groups. Representative heterocyclyl groups include, but are not limited to pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)3 wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃)₂ and the like.

The term “optionally substituted,” or “optional substituents,” as used herein, means that the groups in question are either unsubstituted or substituted with one or more of the substituents specified. When the groups in question are substituted with more than one substituent, the substituents may be the same or different. When using the terms “independently,” “independently are,” and “independently selected from” mean that the groups in question may be the same or different. Certain of the herein defined terms may occur more than once in the structure, and upon such occurrence each term shall be defined independently of the other.

The compounds described herein may contain one or more chiral centers, or may otherwise be capable of existing as multiple stereoisomers. It is to be understood that in one embodiment, the invention described herein is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

The term “prodrug” means a derivative of a compound that can hydrolyze, oxidize, or otherwise react under biological conditions (in vitro or in vivo) to provide an active compound, particularly a compound of the invention. Examples of prodrugs include, but are not limited to, derivatives and metabolites of a compound of the invention that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Specific prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6th ed. (Donald J. Abraham ed., 2001, Wiley) and Design and Application of Prodrugs (H. Bundgaard ed., 1985, Harwood Academic Publishers GmbH).

Further, in each of the foregoing and following embodiments, it is to be understood that the formulae include and represent not only all pharmaceutically acceptable salts of the compounds, but also include any and all hydrates and/or solvates of the compound formulae or salts thereof. It is to be appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non- crystalline and/or amorphous forms of the compounds.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. Ire cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg. The dosages may be single or divided, and may administered according to a wide variety of protocols, including q.d. (once a day), b.i.d. (twice a day), t.i.d. (three times a clay), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in particular a human being.

In some illustrative embodiments, this invention is related to a compound of formula (I)

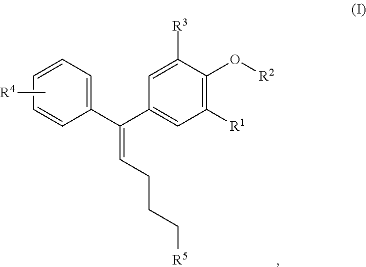

or a pharmaceutically acceptable salt, hydrate, prodrug, polymorph, or solvate thereof, wherein
R¹ is hydrogen, an alkyl, alkenyl, alkynyl, heteroalkyl, or heteroalkenyl;
R² is an alkyl or acyl;
R³ is an acyl or an imidoyl;
R⁴ represents five substituents each independently selected from the group consisting of hydrogen, halo, azido, cyano, nitro, hydroxy, amino, thio, acyl, acyloxy, alkyl, alkenyl, heteroalkyl, heteroalkenyl, heterocyclyl, and aryl, each of which is optionally substituted; or any two adjacent substituents that are taken together with the attached carbons to form an optionally substituted heterocycle and each of other three substitutents is defined as above; and
R⁵ is acyl, alkyl, alkenyl, heteroalkyl, heteroalkenyl, heterocyclyl, cycloalkyl, cycloalkenyl, cycloheteroalkyl, cycloheteroalkenyl, aryl, arylalkyl, or arylalkenyl, each of which is optionally substituted.

In some preferred embodiments, the compound of this invention is represented by formula (I) wherein R¹ is a C₁-C₆ alkyl.

In some preferred embodiments, the compound of this invention is represented by formula (I) wherein R² is a C₁-C₆ alkyl.

In some preferred embodiments, the compound of this invention is represented by formula (I) wherein R³ is an N-alkoxyimidoyl halide.

In some preferred embodiments, the compound of this invention is represented by formula (I) wherein R³ is an acyl thioester and R⁵ is an N-alkoxyimidoyl halide.

In some preferred embodiments, the compound of this invention is represented by formula (I) wherein R⁴ represents five substituents wherein one of the five substituents is a cyano and other four are hydrogen.

In some preferred embodiments, the compound of this invention is represented by formula (I) wherein R⁴ represents five substituents wherein two adjacent substituents are taken together with the attached carbons/heteroatoms to form an optionally substituted cyclic or heterocyclic moiety.

In some preferred embodiments, the compound of this invention is represented by formula (I) wherein R⁵ is an N-alkoxyimidoyl halide.

In some preferred embodiments, the compound of this invention is selected from the group consisting of

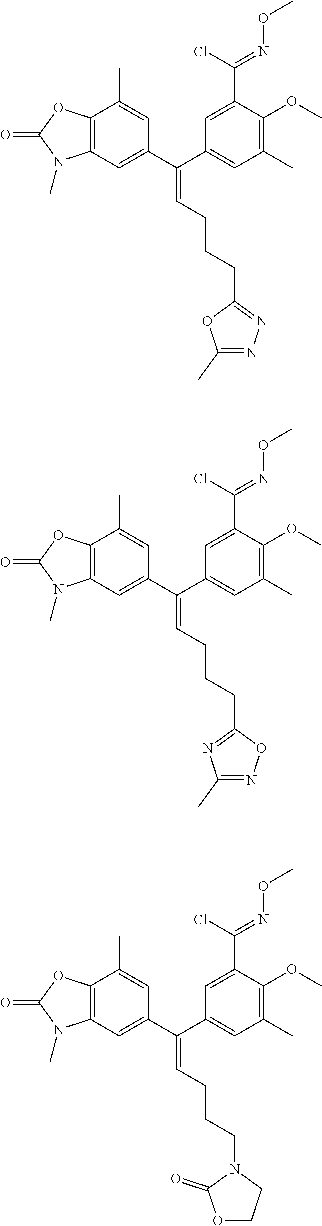 [SMILES: CO/N=C(\Cl)c1cc(/C(=C\CCCc2nnc(C)o2)c2cc(C)c3oc(=O)n(C)c3c2)cc(C)c1OC ; CO/N=C(\Cl)c1cc(/C(=C\CCCc2nc(C)no2)c2cc(C)c3oc(=O)n(C)c3c2)cc(C)c1OC ; CO/N=C(\Cl)c1cc(/C(=C\CCCN2CCOC2=O)c2cc(C)c3oc(=O)n(C)c3c2)cc(C)c1OC]

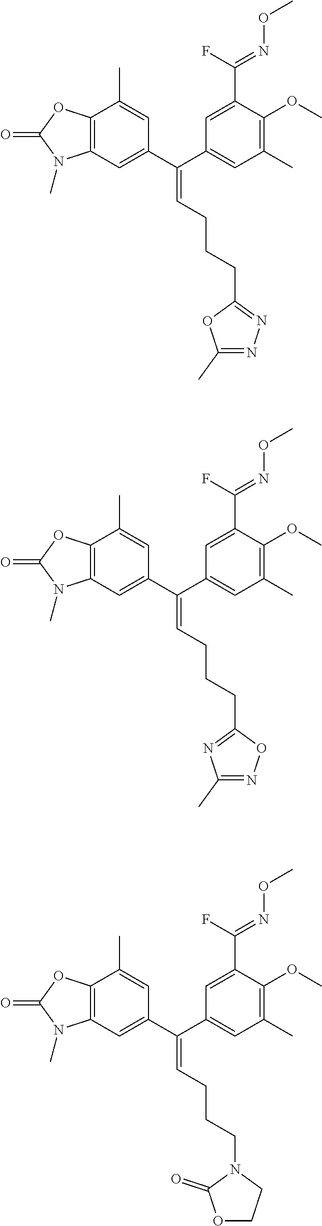 [SMILES: CO/N=C(\F)c1cc(/C(=C\CCCc2nnc(C)o2)c2cc(C)c3oc(=O)n(C)c3c2)cc(C)c1OC ; CO/N=C(\F)c1cc(/C(=C\CCCc2nc(C)no2)c2cc(C)c3oc(=O)n(C)c3c2)cc(C)c1OC ; CO/N=C(\F)c1cc(/C(=C\CCCN2CCOC2=O)c2cc(C)c3oc(=O)n(C)c3c2)cc(C)c1OC]

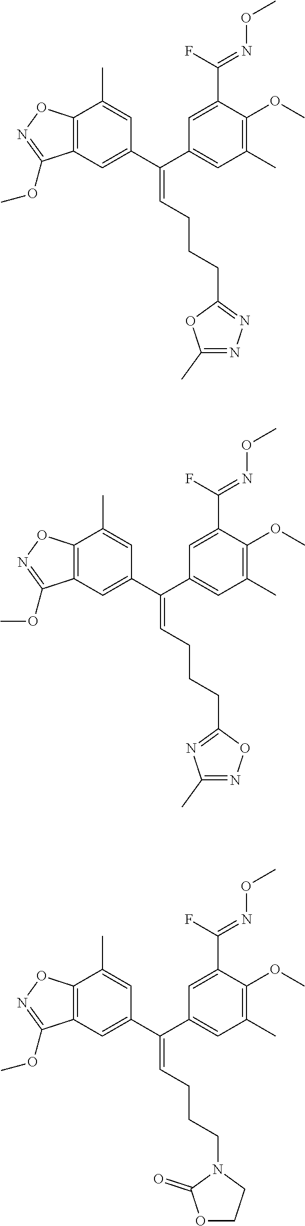

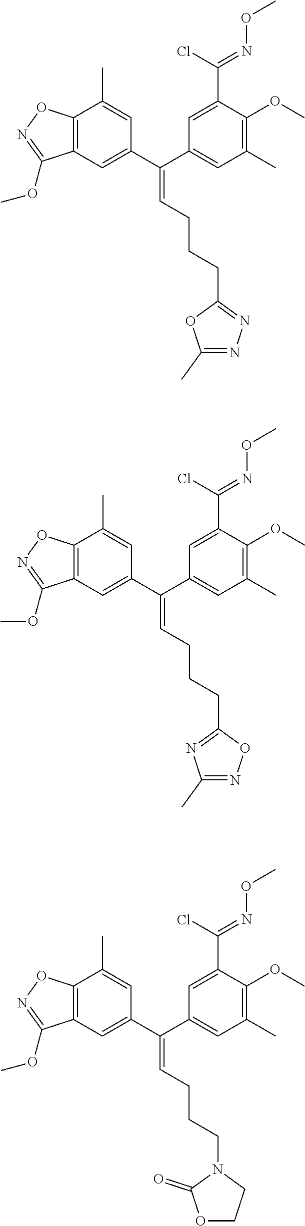

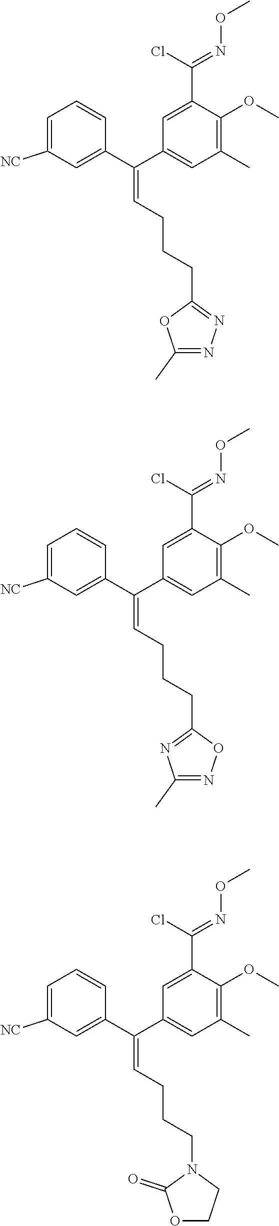 [SMILES: CO/N=C(\Cl)c1cc(/C(=C\CCCc2nnc(C)o2)c2cccc(C#N)c2)cc(C)c1OC ; CO/N=C(\Cl)c1cc(/C(=C\CCCc2nc(C)no2)c2cccc(C#N)c2)cc(C)c1OC ; CO/N=C(\Cl)c1cc(/C(=C\CCCN2CCOC2=O)c2cccc(C#N)c2)cc(C)c1OC]

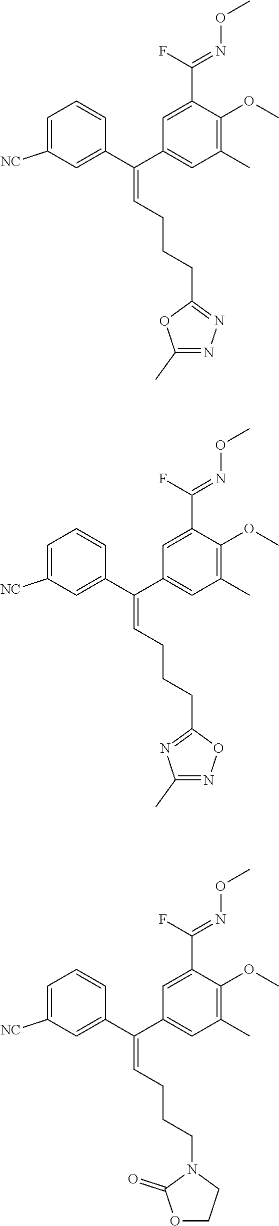 [SMILES: CO/N=C(\F)c1cc(/C(=C\CCCc2nnc(C)o2)c2cccc(C#N)c2)cc(C)c1OC ; CO/N=C(\F)c1cc(/C(=C\CCCc2nc(C)no2)c2cccc(C#N)c2)cc(C)c1OC ; CO/N=C(\F)c1cc(/C(=C\CCCN2CCOC2=O)c2cccc(C#N)c2)cc(C)c1OC]

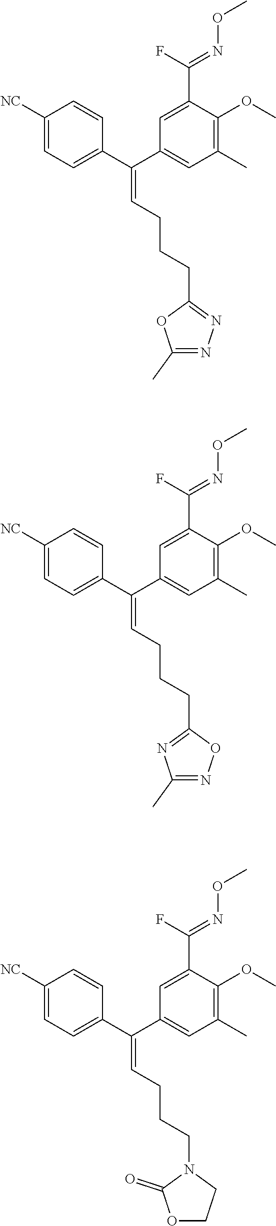 [SMILES: CO/N=C(\F)c1cc(/C(=C\CCCc2nnc(C)o2)c2ccc(C#N)cc2)cc(C)c1OC ; CO/N=C(\F)c1cc(/C(=C\CCCc2nc(C)no2)c2ccc(C#N)cc2)cc(C)c1OC ; CO/N=C(\F)c1cc(/C(=C\CCCN2CCOC2=O)c2ccc(C#N)cc2)cc(C)c1OC]

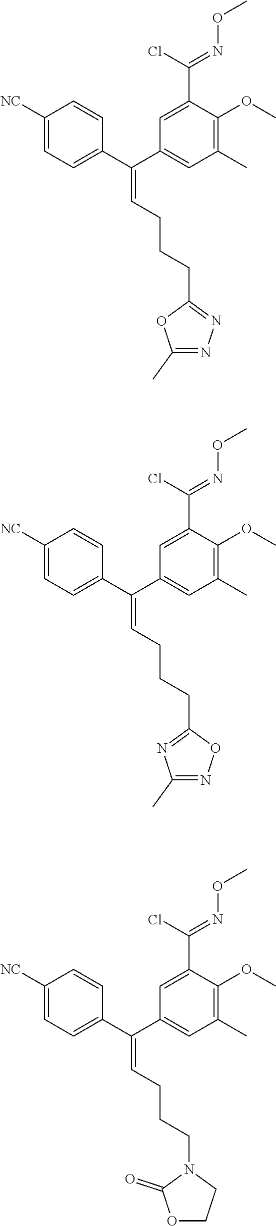 [SMILES: CO/N=C(\Cl)c1cc(/C(=C\CCCc2nnc(C)o2)c2ccc(C#N)cc2)cc(C)c1OC ; CO/N=C(\Cl)c1cc(/C(=C\CCCc2nc(C)no2)c2ccc(C#N)cc2)cc(C)c1OC ; CO/N=C(\Cl)c1cc(/C(=C\CCCN2CCOC2=O)c2ccc(C#N)cc2)cc(C)c1OC]

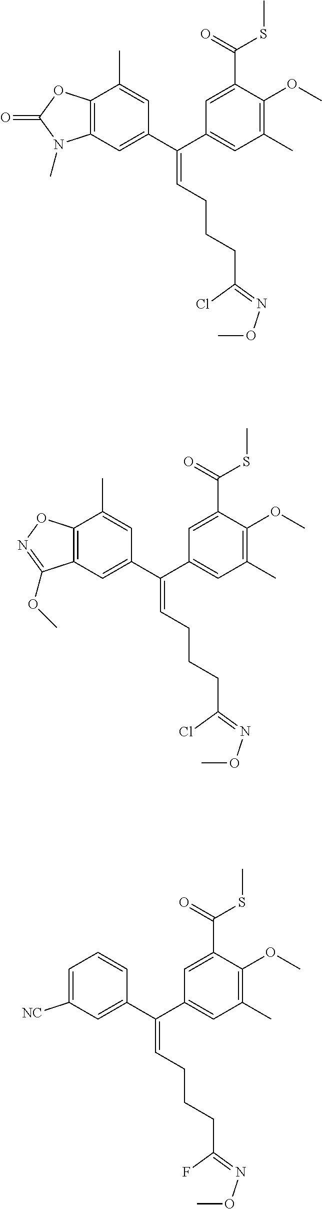 [SMILES: CO/N=C(\Cl)CCC/C=C(\c1cc(C)c(OC)c(C(=O)SC)c1)c1cc(C)c2oc(=O)n(C)c2c1 ; CO/N=C(\Cl)CCC/C=C(\c1cc(C)c(OC)c(C(=O)SC)c1)c1cc(C)c2onc(OC)c2c1 ; CO/N=C(\F)CCC/C=C(/c1cccc(C#N)c1)c1cc(C)c(OC)c(C(=O)SC)c1]

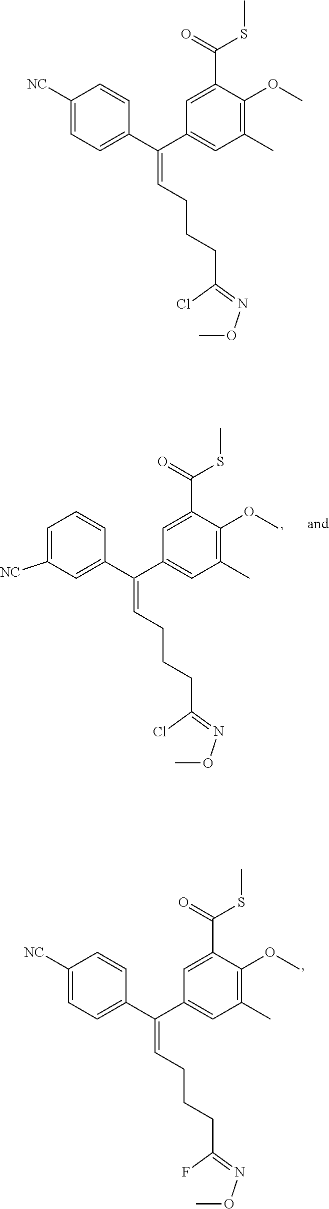 [SMILES: CO/N=C(\Cl)CCC/C=C(/c1ccc(C#N)cc1)c1cc(C)c(OC)c(C(=O)SC)c1 ; CO/N=C(\Cl)CCC/C=C(/c1cccc(C#N)c1)c1cc(C)c(OC)c(C(=O)SC)c1 ; CO/N=C(\F)CCC/C=C(/c1ccc(C#N)cc1)c1cc(C)c(OC)c(C(=O)SC)c1]

or a pharmaceutically acceptable salt, hydrate, prodrug, polymorph, or solvate thereof.

In some preferred embodiments, the compound of this invention includes

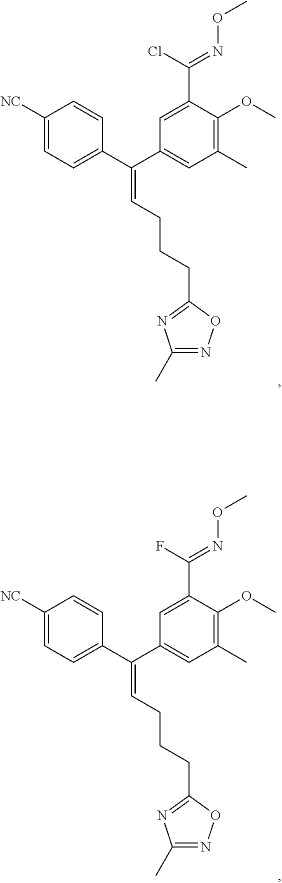 [SMILES: CO/N=C(\Cl)c1cc(/C(=C\CCCc2nc(C)no2)c2ccc(C#N)cc2)cc(C)c1OC ; CO/N=C(\F)c1cc(/C(=C\CCCc2nc(C)no2)c2ccc(C#N)cc2)cc(C)c1OC]

or a pharmaceutically acceptable salt, hydrate, prodrug, polymorph, or solvate thereof.

It is to be understood that all possible combinations of the various genera and subgenera of each of R¹, R², R³, R⁴, and R⁵ in formula (I) described herein represent additional illustrative embodiments of compounds of the invention described herein. It is also to be understood that each of those additional illustrative embodiments of compounds may be used in any of the compositions, methods, and/or uses described herein.

In some embodiments, this invention is related to a pharmaceutical composition comprising a compound represented by formula (I), or a pharmaceutically acceptable salt thereof, together with one or more diluents, excipients or carriers.

In some embodiments, this invention is related to a pharmaceutical composition comprising a compound represented by formula (I), or a pharmaceutically acceptable salt thereof, in combination with one or more other therapeutically active compounds by the same or different modes of action, together with one or more diluents, excipients or carriers.

In some embodiments, this invention is related to a method for treatment of a patient with HIV infection comprising administration of a therapeutically effective amount of a compound represented by formula (I), or a pharmaceutically acceptable salt thereof, together with one or more diluents, excipients or carriers, to the patient in need of relief from said infection.

In some embodiments, this invention is related to a method for treatment of a patient with HIV infection comprising administration of a therapeutically effective amount of a compound represented by formula (I), or a pharmaceutically acceptable salt thereof, in combination with one or more other therapeutically active compounds by the same or different modes of action, together with one or more diluents, excipients or carriers, to the patient in need of relief from said infection.

In some other embodiment, this invention is related to a method for the treatment of a patient with HIV infection comprising administration to the patient in need of relief from said infection a therapeutically effective amount of a compound of formula (I)

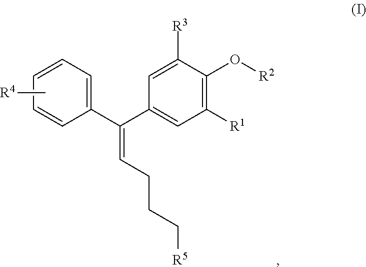

or a pharmaceutically acceptable salt, hydrate, prodrug, polymorph, or solvate thereof, wherein
R¹ is hydrogen, an alkyl, alkenyl, alkynyl, heteroalkyl, or heteroalkenyl;
R² is an alkyl or acyl;
R³ is an acyl or an imidoyl;
R⁴ represents five substituents each independently selected from the group consisting of hydrogen, halo, azido, cyano, nitro, hydroxy, amino, thio, acyl, acyloxy, alkyl, alkenyl, heteroalkyl, heteroalkenyl, heterocyclyl, and aryl, each of which is optionally substituted; or any two adjacent substituents that are taken together with the attached carbons to form an optionally substituted heterocycle and each of other three substitutents is defined as above; and
R⁵ is acyl, alkyl, alkenyl, heteroalkyl, heteroalkenyl, heterocyclyl, cycloalkyl, cycloalkenyl, cycloheteroalkyl, cycloheteroalkenyl, aryl, arylalkyl, or arylalkenyl, each of which is optionally substituted.

In some preferred embodiments, this present invention is related to a method for the treatment of a patient with HIV infection wherein the method comprises the step of administrating a therapeutic effective amount of compound

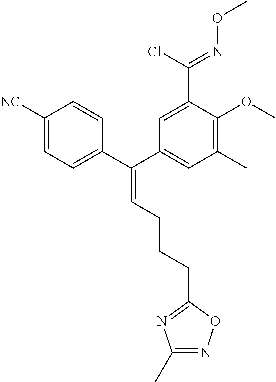 [SMILES: CO/N=C(\Cl)c1cc(/C(=C\CCCc2nc(C)no2)c2ccc(C#N)cc2)cc(C)c1OC]

or a pharmaceutically acceptable salt, hydrate, prodrug, polymorph, or solvate thereof, together with one or more carriers, excipients, or diluents, to the patient in need of relief from said infection.

In some preferred embodiments, this present invention is related to a method for the treatment of a patient with HIV infection wherein the method comprises the step of administrating a therapeutic effective amount of compound

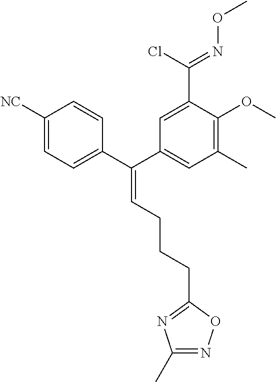 [SMILES: CO/N=C(\Cl)c1cc(/C(=C\CCCc2nc(C)no2)c2ccc(C#N)cc2)cc(C)c1OC]

or a pharmaceutically acceptable salt, hydrate, prodrug, polymorph, or solvate thereof, and one or more other therapeutically effective compounds of the same or different mode of action, together with one or more carriers, excipients, or diluents, to the patient in need of relief from said infection.

In some preferred embodiments, this present invention is related to a method for the treatment of a patient with HIV infection wherein the method comprises the step of administrating a therapeutic effective amount of compound

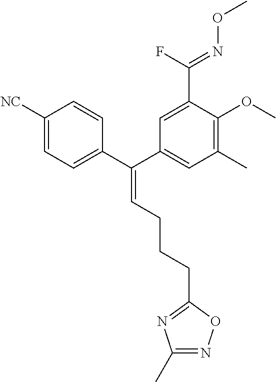 [SMILES: CO/N=C(\F)c1cc(/C(=C\CCCc2nc(C)no2)c2ccc(C#N)cc2)cc(C)c1OC]

or a pharmaceutically acceptable salt, hydrate, prodrug, polymorph, or solvate thereof, together with one or more carriers, excipients, or diluents, to the patient in need of relief from said infection.

In some preferred embodiments, this present invention is related to a method for the treatment of a patient with HIV infection wherein the method comprises the step of administrating a therapeutic effective amount of compound

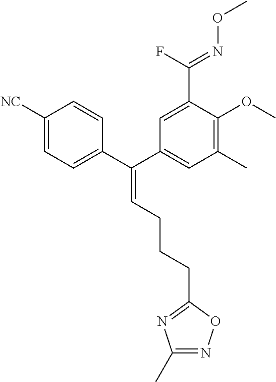 [SMILES: CO/N=C(\F)c1cc(/C(=C\CCCc2nc(C)no2)c2ccc(C#N)cc2)cc(C)c1OC]

or a pharmaceutically acceptable salt, hydrate, prodrug, polymorph, or solvate thereof, and one or more other therapeutically effective compounds of the same or different mode of action, together with one or more carriers, excipients, or diluents, to the patient in need of relief from said infection.

In some embodiments, pharmaceutical compositions containing one or more of the compounds are also described herein for treating a patient with AIDS. It is to be understood that the compositions may include other component and/or ingredients, including other therapeutically active compounds with the same or different modes of action.

The following non-limiting exemplary embodiments are included herein to further illustrate the invention. These exemplary embodiments are not intended and should not be interpreted to limit the scope of the invention in any way.

Chemistry. The structural details of target compounds disclosed in this inventions are shown in Scheme 1. The syntheses of ADAMs for the present studies are facilitated by several prior investigations that pioneered pathways to the modular fragments and established practical methods of connecting them (Scheme 2). The aryl halides 30 and 34 and alkyne 31 synthons were connected using Pd-catalyzed reactions (Sonogashira coupling, hydrostannation, and Stille coupling).

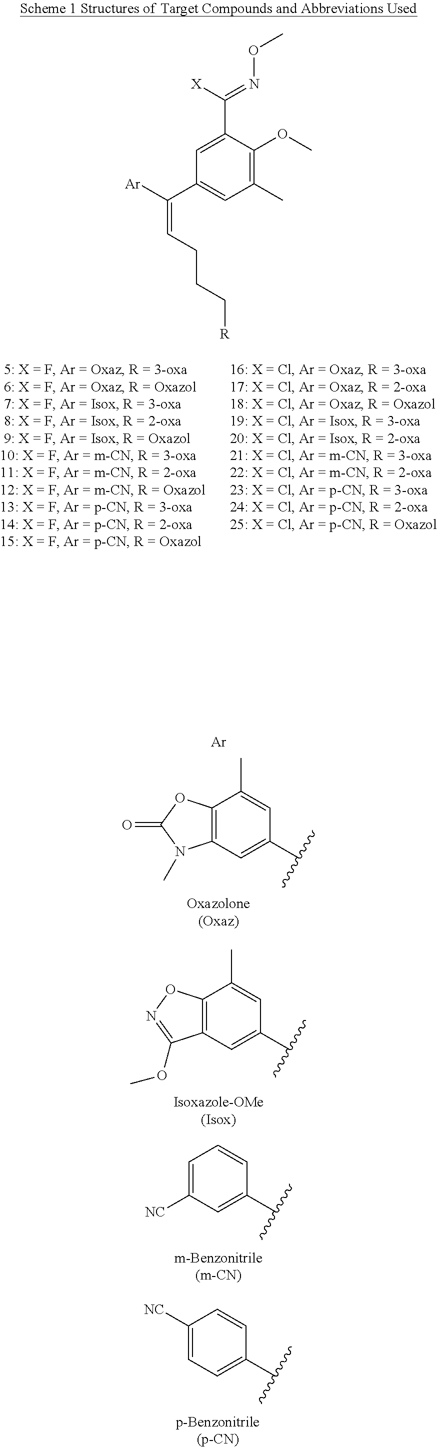

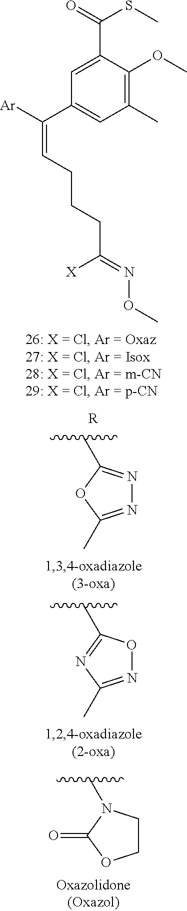

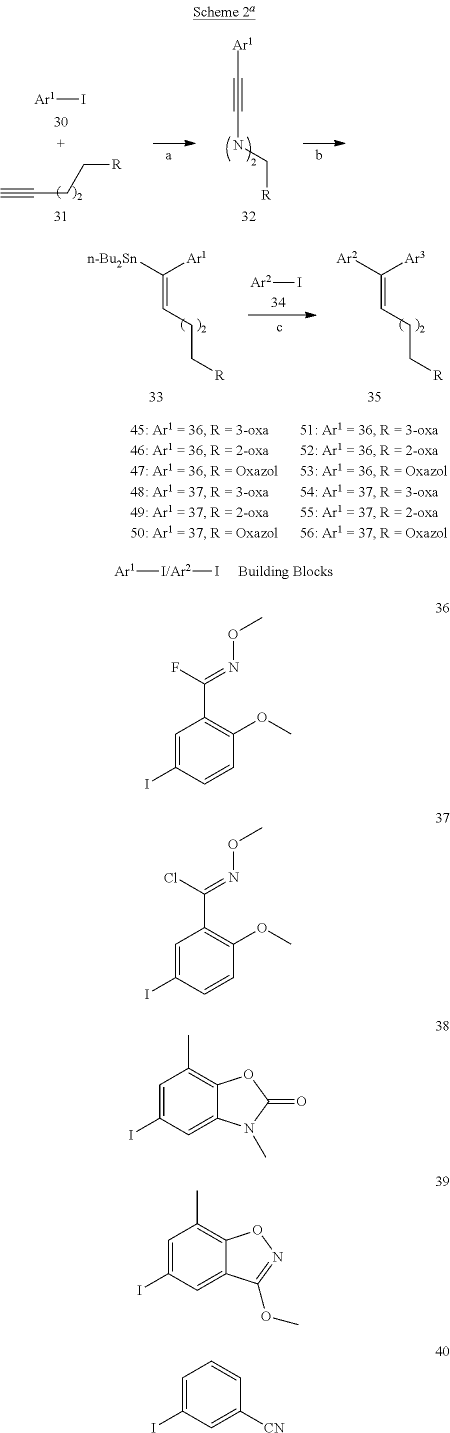

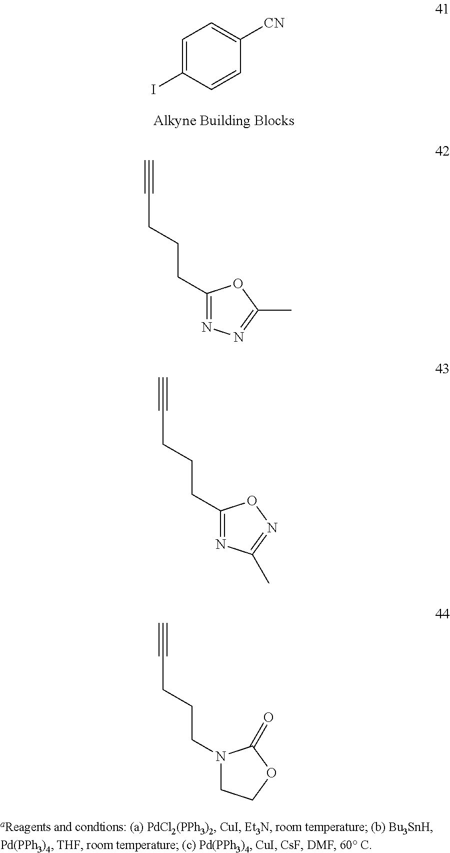

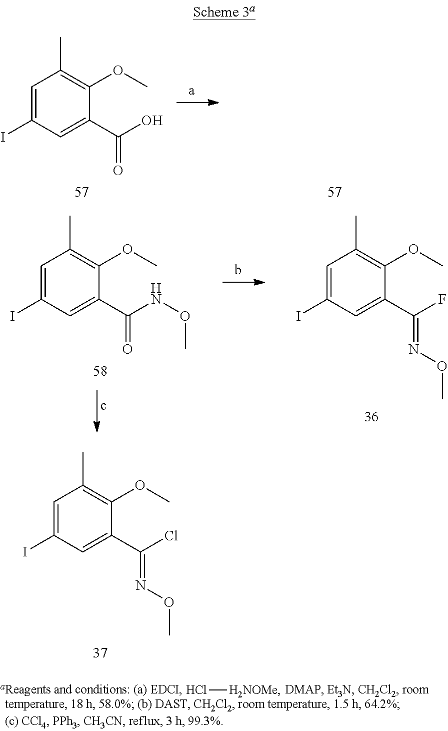

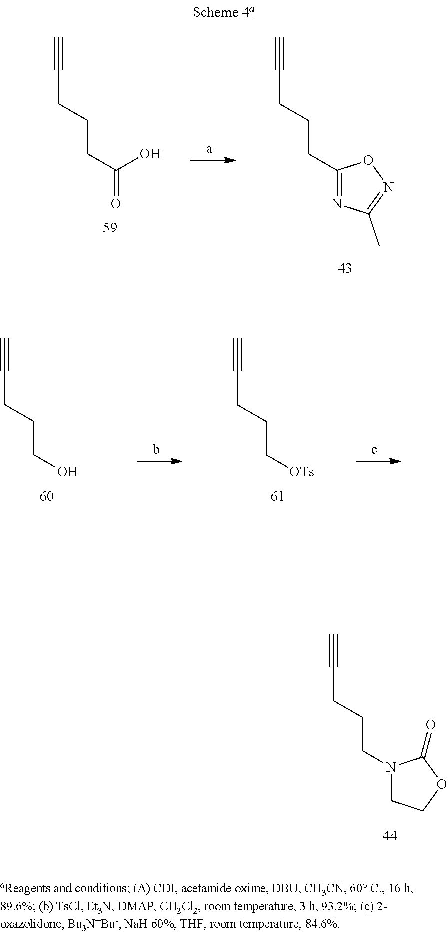

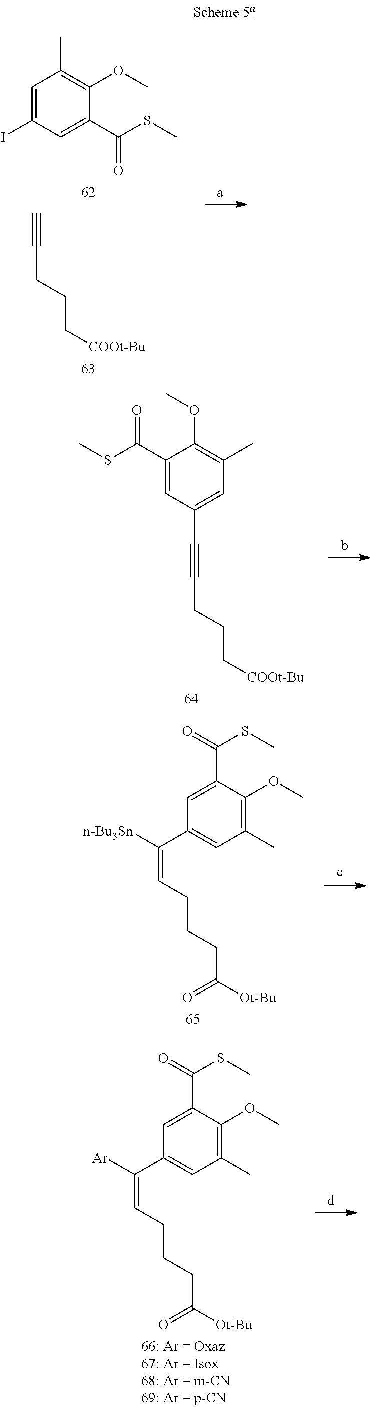

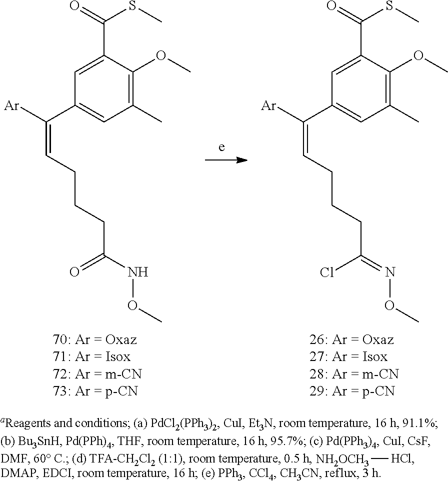

The syntheses of intermediates 36 and 37, which were required for the Sonogashira couplings with alkynes 31, are presented in Scheme 3. Reaction of the carboxylic acid 57 with EDCI and methoxyamine afforded the methoxyamide 58, which on treatment with CC₁₄ and PPh₃ afforded the methoxyimidoyl chloride 37 in quantitative yield. Synthesis of N-methoxyimidoyl fluoride 36 was carried out in 65% yield by reaction of N-methoxyamide 58 with bis(diethyl)aminosulfur trifluoride (DAST). During the preparation of alkynes for the Sonogashira couplings, alkyne 43 was produced in 80% yield by treatment of the carboxylic acid 59 with acetamide oxime and CDI, followed by cyclization of the resulting intermediate with DBU (Scheme 4). Alkyne 44 was prepared in 84% yield by S_{N}2 reaction of 2-oxazolidone with the tosylate 61 of 5-hexyn-1-ol (60).

ADAMs 26-29, having an N-methoxyimidoyl chloride at the end of the side chain, were synthesized by reaction of the corresponding N-methoxyamides 70-73 with PPh₃-CCl₄ in the final stage because it was reported that N-alkoxyimidoyl halides can undergo palladium-catalyzed coupling reactions (Scheme 5). General procedures of ADAM synthesis were applied to prepare intermediates 66-69. Subsequently, the t-butyl ester in the side chain was transformed into N-methoxy amides 70-73, which were reacted with PPh₃-CC₁₄ to afford 26-29.

RT Inhibition Assay. Inhibition of purified recombinant reverse transcriptase was measured by the incorporation of [³²P]GTP into poly(rC)/oligo(dG) homopolymer template primers, as previously described (Cushman, M., et al., J. Med. Chem. 1996, 39, 3217-3227).

In Vitro Antiviral Assay. Evaluation of the antiviral activity of compounds against HIV- 1RF infection in CEM-SS cells was performed using the XTT cytoprotection assay, as previously described (Buckheit, R. W. Jr., et al., Antiviral Res. 1995, 26, 117-132). Evaluation of the antiviral activity of compounds against HIV-1_{IIIB} and HIV-2_{ROD} in MT-4 cells was performed using the MTT assay, as previously described (Rice, W. G. and Bader, J. P., Adv. Pharmacol. (San Diego) 1995, 33, 389-438).

Biological Results. The antiviral activities of the ADAMs were evaluated by determining their ability to inhibit the enzymatic activity of HIV-1 RT in vitro (IC₅₀) and protect HIV-infected cells from the cytopathic effects (EC₅₀) of two viral strains (HIV-1RF and HIV-1_{IIIB}). The cytotoxicities (CC₅₀) of the ADAMs were determined on CEM-SS cells and MT-4 cells. The IC₅₀, EC₅₀, and CC₅₀ values and selectivity indices (SI: CC₅₀/EC₅₀ ratio) of ADAMs 5-29 are presented in Table 1.

TABLE 1
Anti-HIV Activity and Cytotoxicity of ADAM Analogues ^{a}

| | | EC₅₀^{c} (μM) | | CC₅₀^{d} (μM) | | SI (CC₅₀/EC₅₀) | |
|---|---|---|---|---|---|---|---|
| | IC₅₀^{b} | HIV-1_{RF} | HIV-1_{IIIB} | CEM-SS | MT-4 | CEM-SS | MT-4 |
| | (μM) | (CEM-SS) | (MT-4) | cells | cells | cells | cells |
| 5 | 3.40 | 0.50 | 0.35 | 6.50 | 26.48 | 13 | 76 |
| 6 | 2.37 | >100 | >7.53 | 5.04 | 7.53 | | |
| 7 | 1.40 | 1.90 | 2.86 | 13.90 | 15.80 | 7 | 6 |
| 8 | 1.85 | 2.04 | 4.07 | 5.39 | 9.66 | 3 | 2 |
| 9 | 46.69 | >100 | >26.06 | 17.75 | 26.06 | | |
| 10 | 37.10 | >100 | >1.09 | 1.80 | 1.07 | | |
| 11 | 5.40 | >100 | >4.04 | 3.50 | 4.04 | | |
| 12 | >100 | >100 | >1.66 | 1.66 | 1.66 | | |
| 13 | 44.40 | >100 | ≧9.00 | 6.70 | 11.37 | | ≦1 |
| 14 | 0.91 | 2.03 | 5.55 | 13.39 | 20.40 | 7 | 4 |
| 15 | >100 | >100 | >21.13 | 6.70 | 21.13 | | |
| 16 | 8.20 | 1.10 | 0.74 | 4.70 | 19.95 | 4 | 27 |
| 17 | 4.19 | 0.51 | 0.50 | 2.37 | 29.37 | 5 | 59 |
| 18 | 62.9 | >100 | >5.19 | 5.99 | 5.19 | | |
| 19 | 33.80 | 5.20 | ≧7.03 | 9.70 | 17.50 | 2 | ≦2 |
| 20 | 8.03 | 1.70 | 4.21 | 11.37 | 21.24 | 7 | 5 |
| 21 | 33.30 | 5.80 | 2.66 | 13.20 | 22.30 | 2 | 6 |
| 22 | >100 | 3.5 | 2.69 | 25.50 | 109.60 | 7 | 41 |
| 23 | 32.50 | 2.60 | 3.64 | 18.10 | 30.23 | 7 | 8 |
| 24 | 0.10 | 2.52 | 2.75 | 16.90 | 97.52 | 7 | 35 |
| 25 | 42.10 | >100 | >6.48 | 6.48 | 6.48 | | |
| 26 | 3.40 | <0.32 | 0.54 | 7.45 | 58.99 | >23 | 109 |
| 27 | 36.40 | 2.42 | 3.27 | 7.20 | 16.27 | 3 | 5 |
| 28 | 45.30 | 1.46 | 2.43 | 75.50 | 142.8 | 52 | 59 |
| 29 | 12.0 | 2.29 | 3.26 | 56.50 | 108.7 | 25 | 33 |

^{a} All data represent mean values of at least two separate experiments.
^{b}Inhibitory activity versus HIV-1 reverse transcriptase with poly(rC):oligo(dG) as the template primer.
^{c}EC₅₀ is the 50% effective concentration for inhibition of cytopathicity of HIV-1_{RF} in CEM-SS cells, or HIV-1_{IIIB} in MT-4 cells.
^{d}CC₅₀ is the cytotoxic concentration for the mock-infected CEM-SS cells or MT-4 cells.

Unless noted otherwise, ¹H NMR spectra were recorded at 270 MHz or 400 MHz, and ¹³C NMR were recorded at 68 MHz or 100 MHz using CDCl₃ as the solvent and TMS as internal standard. Mass spectrometry data were collected on a high-resolution MS instrument or a low-resolution MS instrument using EI or FAB ionization. Elemental analyses were performed in the Microanalytical Laboratory of Josai University. Flash chromatography was performed with 40-50 μm silica gel. Unless otherwise stated, chemicals and solvent were of reagent grade and used as obtained from commercial sources without further purification. All yields refer to yields of isolated compounds. The purities of all biologically tested compounds were determined by HPLC, with the major peak accounting for >96% of the combined total peak area when monitored by a UV detector at 254 nm.

5-Iodo-N,2-dimethoxy-3-methylbenzamide (58). EDCI (1.70 g, 8.87 mmol) was added to a mixture of benzoic acid 57 (1.84 g, 6.30 mmol), HCl—H₂NOMe (74 mg, 8.9 mmol), DMAP (180 mg, 1.47 mmol), and Et₃N (1.65 mL, 11.9 mmol) in CH₂Cl₂ (38 mL). The reaction mixture was stirred for 18 h at room temperature and then diluted with ethyl acetate (75 mL). The organic solution was washed with 5% HCl (2×100 mL), sat. aq NaHCO₃ (2×100 mL), and brine (2×100 mL) and dried over anhydrous Na₂SO₄. After removal of solvent in vacuo, the residue was purified by column chromatography on silica gel using 30% ethyl acetate-hexanes to give the product 58 (1.18 g, 58.3%) as a white crystalline solid: mp 126-128° C. (EtOAc-hexanes). ¹H NMR (270 MHz, CDCl₃) δ 10.18 (s, 1 H), 8.11 (s, 1 H), 7.63 (s, 1 H), 3.88 (s, 3 H), 3.78 (s, 3 H), 2.27 (s, 3 H); ¹³C NMR (68 MHz, CDCl₃) δ 162.1, 155.5, 142.6, 136.8, 133.7, 126.2, 87.7, 63.9, 61.2, 15.4; EIMS m/z (rel intensity) 321 (6.1, M⁺), 275 (100), 260 (7.3), 148 (5.7), 77 (5.0); Anal. Calcd for C₁₀H₁₂INO₃: C, 37.40; H, 3.77; N, 4.36; found: C, 37.43; H, 3.74; N, 4.37.

5-Iodo-N,2-dimethoxy-3-methylbenzenecarboximidoyl Fluoride (36). Bis(diethylamino)sulfur trifluoride (0.82 mL, 6.2 mmol) was added dropwise to a solution of N-methoxyamide 58 (1.00 g, 3.11 mmol) in dry CH₂Cl₂ (27 mL) at 0° C. under argon. The mixture was stirred for 1 h at 0° C., and for 1.5 h at room temperature. After quenching the reaction with sat. aq. NaHCO₃ (15 mL), the mixture was extracted with ethyl acetate (50 mL) and the organic solvent was dried over anhydrous Na₂SO₄. After removal of solvent in vacuo, the residue was purified by column chromatography on silica gel using 0.6% ethyl acetate-hexanes to give the product 36 (641 mg, 63.8%) as a yellow oil: ¹H NMR (400 MHz, CDCl₃) δ 7.71 (d, J=2.0 Hz, 1 H), 7.61 (d, J=2.0 Hz, 1 H), 3.97 (s, 3 H), 3.79 (s, 3 H), 2.27 (s, 3 H); ¹³C NMR (100 MHz, CDCl₃) δ 157.0, 148.2 (d, J_{CF}=324.7 Hz), 142.6, 135.9 (d, J_{CCCF}=4.6 Hz), 135.1, 122.2 (d, J_{CCF}=27.1 Hz), 86.8, 63.2 (d, J_{CONCF}=1.6 Hz), 60.9 (d, J_{COCCCF}=1.6 Hz), 15.3; EIMS m/z (rel intensity) 323 (M+, 45), 277 (100), 150 (93); HRMS (EI) for C₁₀H₁₁FINO₂ calcd 322.9819 (M⁺), found 322.9805.

5-Iodo-N,2-dimethoxy-3-methylbenzenecarboximidoyl Chloride (37). Carbon tetrachloride (3.18 mL, 32.9 mmol) and PPh₃ (2.16 g, 8.22 mmol) were added to a solution of N-methoxyamide 58 (880 mg, 2.74 mmol) in CH₃CN (20 mL). The reaction mixture was stirred for 0.5 h at room temperature and for 3 h at 95° C. After the mixture was evaporated, the residue was purified by column chromatography on silica gel using 10% ethyl acetate-hexanes to give the product 37 (923 mg, 99.2%) as an oil: ¹H NMR (270 MHz, CDCl₃) δ 7.50 (s, 1 H), 7.48 (s, 1 H), 3.99 (d, J=9.5 Hz, 3 H), 3.68 (d, J=9.5 Hz, 3 H), 2.16 (s, 3 H); ¹³C NMR (68 MHz, CDCl₃) δ 156.5, 141.8, 136.8, 134.6, 132.9, 129.0, 86.7, 63.2, 61.3, 16.0; EIMS m/z (rel intensity) 339 (M⁺, 43), 341 (M+₊2, 14), 304 (98), 273 (100), 258 (26); HRMS (EI) for C₁₀H₁₁ClINO₂ calcd 338.9766 (M⁺), found 338.9496.

3-Methyl-5-(pent-4-yn-1-yl)-1,2,4-oxadiazole (43). Acetamide oxime (330 mg, 4.46 mmol) was added to a stirred solution of CDI (723 mg, 4.46 mmol) and 5-hexynoic acid 59 (50 mg, 4.5 mmol) in CH₃CN (7 mL) at room temperature during 30 min under Ar. The mixture was stirred at room temperature for 5.5 h, DBU (0.73 mL, 4.91 mmol) was added, and the mixture was heated at 60° C. at 16 h. The reaction mixture was extracted with CH₂Cl₂ (20 mL×2), washed with H₂O (40 mL), 1M HCl (30 mL), sat. aq NaHCO₃ (20 mL), brine (40 mL), dried over anhydrous Na₂SO₄, and concentrated. The residue was purified by column chromatography on silica gel using 15% CH₂Cl₂-pentane to give the product 43 (600 mg, 89.6%) as an oil: ¹H NMR (400 MHz, CDCl₃) δ 2.93 (t, J=7.3 Hz, 2 H), 2.48 (s, 3 H), 2.32 (dt, J=7.3, 5.6 Hz, 2 H), 1.99 (quint, J=7.3 Hz, 2 H), 2.03 (t, J=5.6 Hz, 1 H); ¹³C NMR (100 MHz, CDCl₃) δ 178.8, 167.0, 82.4, 69.7, 25.1, 25.0, 17.7, 11.5; EIMS m/z (rel intensity) 150 (M⁺, 0.5), 149 (1.7), 121 (14), 98 (100), 94 (28), 67 (25).

Pent-4-yn-1-yl 4-Methylbenzenesulfonate (61). p-Toluenesulfonyl chloride (4.08 g, 21.4 mmol), Et₃N (3.79 mL, 27.4 mmol) and DMAP (130 mg, 1.19 mmol) were added to a solution of 4-pentyn-1-ol 60 (1.00 g, 11.9 mmol) in CH₂Cl₂ (50 mL). After the mixture was stirred for 3 h at room temperature, 5% aq HCl (50 mL) added. The mixture was extracted with CH₂Cl₂ (25 mL×2), and the organic solution was washed brine and dried over anhydrous Na₂SO₄. After removal of solvent in vacuo, the residue was purified by column chromatography on silica gel eluting with 15% ethyl acetate-hexanes to give the product 61 (2.64 g, 93.2%) as a clear oil: ¹H NMR (400 MHz, CDCl₃) δ 7.79 (d, J=8.2 Hz, 2 H), 7.36 (d, J=8.2 Hz, 2 H), 4.14 (t, J=6.5 Hz, 2 H), 2.45 (s, 3 H), 2.25 (dt, J=6.5, 2.8 Hz, 2 H), 1.90 (t, J=2.8 Hz, 1 H), 1.85 (quint, J=6.5 Hz, 2 H); ¹³C NMR (100 MHz, CDCl₃) δ 144.7, 132.7, 129.7, 127.7, 82.0, 69.3, 68.6, 27.5, 21.5, 14.5; EIMS m/z (rel intensity) 238 (M⁺, 0.9), 174 (21), 155 (46), 91 (100), 66 (50), 65 (30); HRMS (EI) for C₁₂H₁₄O₃S calcd 238.0664 (M⁺), found 238.0667.

3-(Pent-4-yn-1-yl)-1,3-oxazolidin-2-one (44). p-Toluenesulfonate 61 (200 mg, 0.84 mmol) was added dropwise to a stirred solution of NaH (60 wt % dispersion in mineral oil, 200 mg, 15.2 mmol), tetra-n-butylammonium iodide (80 mg, 0.76 mmol), and 1,3-oxazolidin-2-one (220 mg, 2.53 mmol) in dry THF (15 mL) for 30 min at 0° C. After the mixture was stirred for 18 h at room temperature, the reaction was quenched. The mixture was extracted with CH₂Cl₂ (25 mL×2) and the organic solution was washed with brine and dried over anhydrous Na₂SO₄. After removal of solvent in vacuo, the residue was purified by column chromatography on silica gel eluting with 50% ethyl acetate-hexanes to 10% MeOH-ethyl acetate to give the product 44 (110 mg, 84.6%) as a yellow oil: ¹H NMR (270 MHz, CDCl₃) δ 4.34 (t, J=8.0 Hz, 2 H), 3.60 (t, J=8.0 Hz, 2 H), 3.38 (t, J=7.2 Hz, 2 H), 2.27 (dt, J=7.2, 2.8 Hz, 2 H), 2.00 (t, J=2.8 Hz, 1 H), 1.81 (quint, J=7.2 Hz, 2 H); ¹³C NMR (68 MHz, CDCl₃) δ 82.8, 69.0, 61.6, 44.7, 43.2, 26.1, 15.8, 14.1; FABMS (glycerine) m/z 154 (MH⁺); HRMS (FAB) for C₈H₁₂NO₂ calcd 154.0868 (Mtr), found 154.0865.

General Procedure (Sonogashira Coupling). A solution of alkyne 31 (1.2 equiv), aryl iodide 30 (1.0 equiv), and Et₃N (2.0 equiv) in dry THF was cooled to 0° C. and degassed for 10 min with argon. Then CuI (0.10 equiv) and PdCl₂(PPh₃)₂ (0.10 equiv) were added to the solution. After stirring for 10-20 h at room temperature under argon, the mixture was evaporated and diluted with ethyl acetate. The mixture was filtered through a small pad of silica gel and concentrated. The residue was purified by column chromatography on silica gel using an ethyl acetate-hexanes to provide the product.

N,2-Dimethoxy-3-methyl-5- [5-(5-methyl-1,3,4-oxadiazol-2-yl)pent-1-yn-1-yl]benzenecarboximidoyl Fluoride (45). The general Sonogashira coupling procedure was followed using alkyne 42 (320 mg, 2.10 mmol), aryl iodide 36 (560 mg, 1.75 mmol), Et₃N (0.50 mL, 3.5 mmol), CuI (40 mg, 0.18 mmol) and PdCl₂(PPh₃)₂ (120 mg, 0.175 mmol) in THF (15 mL). The reaction mixture was stirred for 20 h and the product was purified by column chromatography on silica gel using 50% ethyl acetate-hexanes to give the product 45 (468 mg, 77.5%) as a yellow oil: ¹H NMR (270 MHz, CDCl₃) δ 7.45 (s, 1 H), 7.32 (s, 1 H), 3.97 (s, 3 H), 3.80 (s, 3 H), 3.00 (t, J=7.2 Hz, 2 H), 2.55 (t, J=7.2 Hz, 2 H), 2.50 (s, 3 H), 2.28 (s, 3 H), 2.08 (quint, J=7.2 Hz, 2 H); ¹³C NMR (68 MHz, CDCl₃) δ 166.1, 163.5, 156.5, 148.8 (d, J_{CF}=324.5 Hz), 136.7, 132.6, 130.5 (d, J_{CCCF}=4.5 Hz), 120.1 (d, J_{CCF}=27.9 Hz), 119.1, 88.3, 80.2, 63.1 (d, J_{COCCCF}=2.2 Hz), 60.9 (d, J_{CONCF}=2.2 Hz), 25.2, 24.3, 18.7, 15.8, 10.9; EIMS m/z (rel intensity) 345 (M⁺, 38), 248 (34), 197 (21), 98 (100); HRMS (EI) for C₁₈H₂₀FN₃O₃ calcd 345.1488 (M⁺), found 345.1493.

N,2-Dimethoxy-3-methyl-5-[5-(3-methyl-1,2,4-oxadiazol-5-yl)pent-1-yn-1-yl]benzenecarboximidoyl Fluoride (46). The general Sonogashira coupling procedure was followed using alkyne 43 (459 mg, 3.06 mmol), aryl iodide 36 (823 mg, 2.55 mmol), Et₃N (0.71 mL, 5.1 mmol), CuI (49 mg, 0.26 mmol) and PdCl₂(PPh₃)₂ (179 mg, 0.255 mmol) in THF (12 mL). The reaction mixture was stirred for 20 h and the product was purified by column chromatography on silica gel using 15% ethyl acetate-hexanes to give the product 46 (760 mg, 86.3%) as an oil: ¹H NMR (400 MHz, CDCl₃) δ 7.45 (d, J=1.8 Hz, 1 H), 7.32 (d, J=1.8 Hz, 1 H), 3.97 (s, 3 H), 3.80 (s, 3 H), 3.04 (t, J=7.2 Hz, 2 H), 2.55 (t, J=7.2 Hz, 2 H), 2.38 (s, 3 H), 2.28 (s, 3 H), 2.11 (quint, J=7.2 Hz, 2 H); ¹³C NMR (100 MHz, CDCl₃) δ 178.9, 167.1, 156.7, 149.1 (d, J_{CF}=323.0 Hz), 137.0, 132.8, 130.8 (d, J_{CCCF}=4.4 Hz), 120.4 (d, J_{CCF}=29.0), 111.2, 88.1, 80.5, 63.2, 61.0, 25.4, 25.3, 18.7, 15.9, 11.5; EIMS m/z (rel intensity) 345 (M⁺, 60), 248 (83), 197 (81), 153 (36), 98 (100); HRMS (EI) for C₁₈H₂₀FN₃O₃ calcd 345.1489 (M⁺), found 345.1474.

N,2-Dimethoxy-3-methyl-5-[5-(2-oxo-1,3-oxazolidin-3-yl)pent-1-yn-1-yl]benzenecarboximidoyl Fluoride (47). The general Sonogashira coupling procedure was followed using alkyne 44 (149 mg, 0.972 mmol), aryl iodide 36 (262 mg, 0.810 mmol), Et₃N (0.22 mL, 1.6 mmol), CuI (15 mg, 0.081 mmol) and PdCl₂(PPh₃)₂ (57 mg, 0.081 mmol) in THF (6 mL). The reaction mixture was stirred for 20 h and the product was purified by column chromatography on silica gel using 50% ethyl acetate-hexanes to give the product 47 (214 mg, 75.9%) as a yellow oil: ¹H NMR (270 MHz, CDCl₃) δ 7.45 (d, J=2.2 Hz, 1 H), 7.34 (d, J=2.2 Hz, 1 H), 4.35 (t, J=8.0 Hz, 2 H), 3.97 (s, 3 H), 3.80 (s, 3 H), 3.61 (t, J=8.0 Hz, 2 H), 3.42 (t, J=7.1 Hz, 2 H), 2.45 (t, J=7.1 Hz, 2 H), 2.28 (s, 3 H), 1.87 (quint, J=7.1 Hz, 2 H); ¹³C NMR (68 MHz, CDCl₃) δ 158.3, 156.4, 148.8 (d, J_{CF}=324.8 Hz), 136.8, 132.7, 130.5 (d, J_{CCCF}=4.5 Hz), 120.2 (d, J_{CCF}=28.0 Hz), 119.2, 88.6, 79.8, 63.1 (d, J_{CONCF}=2.0 Hz), 61.7, 60.9 (d, J_{COCCCF}=2.0 Hz), 44.8, 43.5, 26.4, 16.9, 15.9; EIMS m/z (rel intensity) 348 (M⁺, 37), 297 (36), 28.5 (29), 230 (34), 230 (34), 210 (89), 100 (80), 56 (100); HRMS (EI) for C₁₈H₂₁FN₂O₄ calcd 348.1485 (M⁺), found 348.1486.

N,2-Dimethoxy-3-methyl-5-[5-(5-methyl-1,3,4-oxadiazol-2-yl)pent-1-yn-1-yl]benzenecarboximidoyl Chloride (48). The general Sonogashira coupling procedure was followed using alkyne 42 (160 mg, 1.07 mmol), aryl iodide 37 (303 mg, 0.892 mmol), Et₃N (0.23 mL, 1.8 mmol), CuI (20 mg, 0.089 mmol) and PdCl₂(PPh₃)₂ (60 mg, 0.089 mmol) in THF (7 mL). The reaction mixture was stirred for 20 h and the product was purified by column chromatography on silica gel using 50% ethyl acetate-hexanes to give the product 48 (320 mg, 99.0%) as a yellow oil: ¹H NMR (270 MHz, CDCl₃) δ 7.32 (d, J=2.0 Hz, 1 H), 7.28 (d, J=2.0 Hz, 1 H), 4.09 (s, 3 H), 3.79 (s, 3 H), 3.00 (t, J=7.2 Hz, 2 H), 2.54 (t, J=7.2 Hz, 2 H), 2.50 (s, 3 H), 2.28 (s, 3 H), 2.07 (quint, J=7.2 Hz, 2 H); ¹³C NMR (68 MHz, CDCl₃) δ 166.1, 163.5, 156.0, 136.1, 133.6, 132.1, 131.5, 127.1, 118.9, 88.1, 80.4, 63.0, 61.3, 25.3, 24.3, 18.8, 16.1, 11.0; EIMS m/z (rel intensity) 361 (M⁺, 42), 363 (M⁺+2, 15), 294 (26), 266 (12), 264 (29), 197 (37), 98 (100); HRMS (EI) for C₁₈H₂₀³⁵ClN₃O₃ calcd 361.1193 (M⁺), found 361.1198, C₁₈H₂₀³⁷ClN₃O₃ calcd 363.1164 (M⁺+2), found 361.1176.

N,2-Dimethoxy-3-methyl-5-[5-(3-methyl-1,2,4-oxadiazol-5-yl)pent-1-yn-1-yl]benzenecarboximidoyl Chloride (49). The general Sonogashira coupling procedure was followed using alkyne 43 (360 mg, 2.40 mmol), aryl iodide 37 (679 mg, 2.00 mmol), Et₃N (0.56 mL, 6.0 mmol), CuI (38 mg, 0.20 mmol) and PdCl₂(PPh₃)₂(140 mg, 0.200 mmol) in THF (12 mL). The reaction mixture was stirred for 20 h and the product was purified by column chromatography on silica gel using 15% ethyl acetate-hexanes to give the product 49 (720 mg, 99.6%) as an oil: ¹H NMR (400 MHz, CDCl₃) δ 7.32 (d, J=2.0 Hz, 1 H), 7.29 (d, J=2.0 Hz, 1 H), 4.09 (s, 3 H), 3.79 (s, 3 H), 3.04 (t, J=7.2 Hz, 2 H), 2.55 (t, J=7.2 Hz, 2 H), 2.38 (s, 3 H), 2.28 (s, 3 H), 2.10 (quint, J=7.2 Hz, 2 H); ¹³C NMR (100 MHz, CDCl₃) δ 178.9, 167.0, 156.2, 136.3, 133.8, 132.3, 131.7, 127.2, 119.0, 88.0, 80.6, 63.1, 61.3, 25.4, 25.3, 18.7, 16.0, 11.5; EIMS m/z (rel intensity) 361 (M⁺, 29), 363 (M⁺+2, 10), 294 (34), 264 (37), 197 (85), 184 (43), 98 (100); HRMS (EI) for C₁₈H₂₀³⁵ClN₃O₃ calcd 361.1193 (M⁺), found 361.1170, C₁₈H₂₀³⁵ClN₃O₃ calcd 363.1164 (M⁺+2), found 363.1136.

N,2-Dimethoxy-3-methyl-5-[5-(2-oxo-1,3-oxazolidin-3-yl)pent-1-yn-1-yl]benzenecarboximidoyl Chloride (50). The general Sonogashira coupling procedure was followed using alkyne 44 (150 mg, 0.980 mmol), aryl iodide 37 (227 mg, 0.817 mmol), Et₃N (0.22 mL, 1.6 mmol), CuI (15 mg, 0.082 mmol) and PdCl₂(PPh₃)₂ (45 mg, 0.082 mmol) in THF (6 mL). The reaction mixture was stirred for 20 h and the product was purified by column chromatography on silica gel using 50% ethyl acetate-hexanes to give the product 50 (211 mg, 72.3%) as a yellow oil: ¹H NMR (400 MHz, CDCl₃) δ 7.32 (d, J=2.0 Hz, 1 H), 7.30 (d, J=2.0 Hz, 1 H), 4.32 (t, J=8.1 Hz, 2 H), 4.09 (s, 3 H), 3.78 (s, 3 H), 3.61 (t, J=8.1 Hz, 2 H), 3.41 (t, J=7.0 Hz, 2 H), 2.47 (t, J=7.0 Hz, 2 H), 2.28 (s, 3 H), 1.86 (quint, J=7.0 Hz, 2 H); ¹³C NMR (100 MHz, CDCl₃) δ 158.2, 155.9, 136.0, 133.5, 132.1, 131.3, 127.0, 118.8, 88.3, 79.7, 62.8, 61.4, 61.0, 44.5, 43.3, 26.1, 16.6, 15.8; EIMS m/z (rel intensity) 364 (M⁺, 20), 366 (M⁺+2, 6.7), 297 (32), 279 (12), 277 (36), 210 (100), 197 (21), 184 (17), 100 (43); HRMS (EI) for C₁₈H₂₁³⁵ClN₂O₄ calcd 364.1190 (M⁺), found 364.1176, C₁₈H₂₁³⁷ClN₂O₄ calcd 366.1160 (M⁺+2), found 366.1156.

tert-Butyl 6-{4-Methoxy-3-methyl-5-[methylsulfanyl)carbonyl]phenyl}hex-5-ynoate (64). The general Sonogashira coupling procedure was followed using alkyne 63 (2.00 g, 11.9 mmol), aryl iodide 62 (3.20 g, 11.9 mmol), Et₃N (2.77 mL, 19.9 mmol), CuI (227 mg, 1.19 mmol) and PdCl₂(PPh₃)₂ (697 mg, 0.993 mmol) in THF (65 mL). The reaction mixture was stirred for 16 h and the product was purified by column chromatography on silica gel using 5% ethyl acetate-hexanes to give the product 64 (3.28 g, 91.1%) as a colorless oil: ¹H NMR (400 MHz, CDCl₃) δ 7.58 (d, J=2.2 Hz, 1 H), 7.36 (d, J=2.2 Hz, 1 H), 3.80 (s, 3 H), 2.49-2.37 (m, 7 H), 2.28 (s, 3 H), 1.87 (quint, J=7.0 Hz, 2 H), 1.46 (s, 9 H); ¹³C NMR (100 MHz, CDCl₃) δ 191.3, 172.0, 155.5, 137.4, 132.3, 131.4, 129.8, 119.1, 89.1, 80.0, 79.7, 61.6, 34.2, 27.9, 23.9, 18.7, 15.8, 12.3; EIMS m/z (rel intensity) 362 (M⁺, 2.2), 315 (21), 306 (18), 259 (100), 128 (10); HRMS (EI) for C₂₀H₂₆O₄S calcd 362.1551 (M⁺), found 362.1563.

General Procedure (Hydrostannation). n-Bu₃SnH (1.50 equiv) was added to a solution of alkyne (1.00 equiv) and Pd(PPh₃)₄ (0.10 equiv) in anhydrous THF dropwise at 0° C. under argon atmosphere and the reaction mixture was allowed to stir for 16-23 h at ambient temperature. The mixture was concentrated under reduced pressure and diluted with EtOAc (50 mL). The suspension was filtered through a small pad of silica gel. The filtrate was concentrated under reduced pressure and the residue was purified by column chromatography on silica gel using an ethyl acetate-hexanes to give the product.

N,2-Dimethoxy-3-methyl-5-[(1E)-5-(5-methyl-1,3,4-oxadiazol-2-yl)-1-(tributylstannanyl)pent-1-en-1-yl]benzenecarboximidoyl Fluoride (51). The general hydrostannation procedure was followed using alkyne 45 (470 mg, 1.35 mmol), Pd(PPh₃)₄ (160 mg, 0.135 mmol) and n-Bu₃SnH (0.54 mL, 2.0 mmol) in THF (15 mL). The reaction mixture was stirred for 23 h and the product was purified by column chromatography on silica gel using 33% ethyl acetate-hexanes to give the product 51 (692 mg, 80.6%) as a yellow oil: ¹H NMR (270 MHz, CDCl₃) δ 6.94 (s, 1 H), 6.82 (s, 1 H), 5.74 (t, J=7.3 Hz, 1 H), 3.97 (s, 3 H), 3.80 (s, 3 H), 2.74 (t, J=7.3 Hz, 2 H), 2.47 (s, 3 H), 2.28 (s, 3 H), 2.19−2.10 (m, 2 H), 1.83 (quint, J=7.3 Hz, 2 H), 1.52-1.37 (m, 6 H), 1.33-1.19 (m, 6 H), 0.89−0.83 (m, 15 H); ¹³C NMR (68 MHz, CDCl₃) δ 166.7, 163.3, 154.3, 149.6 (d, J_{CF}=324.0 Hz), 145.6, 140.5, 140.2, 132.4, 132.1, 125.4 (d, J_{CCCF}=4.5 Hz), 119.6 (d, J_{CCF}=20.6 Hz), 63.1 (d, J_{CONCF}=2.2 Hz), 60.9 (d, J_{COCCCF}=2.2 Hz), 29.3, 29.0, 27.3, 26.4, 24.8, 16.2, 13.7, 10.9, 10.0; EIMS m/z (rel intensity) 637 (M⁺, 0.03), 580 (100), 578 (75.8), 98 (44.7).

N,2-Dimethoxy-3-methyl-5-[(1E)-5-(3-methyl-1,2,4-oxadiazol-5-yl)-1-(tributylstannanyl)pent-1-en-1-yl]benzenecarboximidoyl Fluoride (52). The general hydrostannation procedure was followed using alkyne 46 (744 mg, 2.15 mmol), Pd(PPh₃)₄ (249 mg, 0.215 mmol) and n-Bu₃SnH (0.86 mL, 3.2 mmol) in THF (12 mL). The reaction mixture was stirred for 20 h and the product was purified by column chromatography on silica gel using 10% ethyl acetate-hexanes to give the product 52 (1.05 g, 76.8%) as an oil: ¹H NMR (270 MHz, CDCl₃) δ 6.94 (d, J=2.1 Hz, 1 H), 6.81 (d, J=2.1 Hz, 1 H), 5.72 (t, J=7.3 Hz, 1 H), 4.00 (s, 3 H), 3.80 (s, 3 H), 2.78 (t, J=7.3 Hz, 2 H), 2.35 (s, 3 H), 2.28 (s, 3 H), 1.92-1.84 (m, 2 H), 1.87 (quint, J=7.3 Hz, 2 H), 1.48-1.37 (m, 6 H), 1.35-1.19 (m, 6 H), 0.95-0.83 (m, 15 H); ¹³C NMR (68 MHz, CDCl₃) δ 179.1, 166.7, 154.3, 149.6 (d, J_{CF}=323.7 Hz), 145.8, 140.3, 140.2, 132.3, 132.0, 125.4 (d, J_{CCCF}=4.5 Hz), 119.7 (d, J_{CCF}=27.4 Hz), 63.2 (d, J_{CONCF}=1.7 Hz), 60.9 (d, J_{COCCCF}=1.7 Hz), 29.2, 29.0, 27.3, 26.4, 25.9, 16.2, 13.7, 11.5.

N,2-Dimethoxy-3-methyl-5-[(1E)-5-(2-oxo-1,3-oxazolidin-3-yl)-1-(tributylstannanyl)pent-1-en-1-yl]benzenecarboximidoyl Fluoride (53). The general hydrostannation procedure was followed using alkyne 47 (200 mg, 0.556 mmol), Pd(PPh₃)₄ (66 mg, 0.057 mmol) and n-Bu₃SnH (0.24 mL, 0.83 mmol) in THF (5 mL). The reaction mixture was stirred for 20 h and the product was purified by column chromatography on silica gel using 66% ethyl acetate-hexanes to give the product 53 (247 mg, 67.9%) as a yellow oil: ¹H NMR (270 MHz, CDCl₃) δ 6.93 (d, J=1.8 Hz, 1 H), 6.83 (d, J=1.8 Hz, 1 H), 5.75 (t, J=7.3 Hz, 1 H), 4.23 (t, J=8.0 Hz, 2 H), 3.96 (s, 3 H), 3.80 (s, 3 H), 3.44 (t, J=8.0 Hz, 2 H), 3.19 (t, J=7.3 Hz, 2 H), 2.29 (s, 3 H), 2.09-2.01 (m, 2 H), 1.61 (quint, J=7.3 Hz, 2 H), 1.48-1.37 (m, 6 H), 1.33-1.19 (m, 6 H), 0.89-0.83 (m, 15 H); ¹³C NMR (68 MHz, CDCl₃) δ 158.1, 154.2, 149.5 (d, J_{CF}=324.5 Hz), 145.0, 140.6, 140.2, 132.4, 132.0, 125.3 (d, J_{CCCF}=4.5 Hz), 119.5 (d, J_{CCF}=27.4 Hz), 62.9 (d, J_{CONCF}=1.8 Hz), 61.5, 60.8 (d, J_{COCCCF}=1.8 Hz), 44.3, 43.7, 28.7, 27.4, 27.2, 16.0, 13.6, 9.90; FABMS (3-nitrobenzyl alcohol) m/z 641 (MH⁺).

N,2-Dimethoxy-3-methyl-5-[(1E)-5-(5-methyl-1,3,4-oxadiazol-2-yl)-1-(tributylstannanyl)pent-1-en-1-yl]benzenecarboximidoyl Chloride (54). The general hydrostannation procedure was followed using alkyne 48 (370 mg, 1.01 mmol), Pd(PPh₃)₄ (120 mg, 0.101 mmol) and n-Bu₃SnH (0.40 mL, 1.4 mmol) in THF (6 mL). The reaction mixture was stirred for 18 h and the product was purified by column chromatography on silica gel using 50% ethyl acetate-hexanes to give the product 54 (530 mg, 80.4%) as a yellow oil: ¹H NMR (270 MHz, CDCl₃) δ 6.83 (d, J=2.2 Hz, 1 H), 6.78 (d, J=2.2 Hz, 1 H), 5.73 (t, J=7.6 Hz, 1 H), 4.09 (s, 3 H), 3.78 (s, 3 H), 2.74 (t, J=7.6 Hz, 2 H), 2.45 (s, 3 H), 2.29 (s, 3 H), 2.20-2.12 (m, 2 H), 1.84 (quint, J=7.6 Hz, 2 H), 1.48-1.37 (m, 6 H), 1.32-1.19 (m, 6 H), 0.89-0.82 (m, 15 H); ¹³C NMR (68 MHz, CDCl₃) δ 166.5, 163.1, 153.5, 145.5, 140.3, 139.9, 134.0, 131.4, 131.3, 126.5, 126.4, 62.7, 61.1, 29.2, 28.9, 27.2, 26.3, 24.6, 16.2, 13.6, 10.8, 9.9; FABMS (3-nitrobenzyl alcohol) m/z 654 (MH⁺).

N,2-Dimethoxy-3-methyl-5-[(1E)-5-(3-methyl-1,2,4-oxadiazol-5-yl)-1-(tributylstannanyl)pent-1-en-1-yl]benzenecarboximidoyl Chloride (55). The general hydrostannation procedure was followed using alkyne 49 (737 mg, 2.04 mmol), Pd(PPh₃)₄ (235 mg, 0.204 mmol) and n-Bu₃SnH (0.81 mL, 3.1 mmol) in THF (10 mL). The reaction mixture was stirred for 20 h and the product was purified by column chromatography on silica gel using 10% ethyl acetate-hexanes to give the product 55 (934 mg, 70.1%) as a oil: ¹H NMR (400 MHz, CDCl₃) δ 6.82 (d, J=2.2 Hz, 1 H), 6.77 (d, J=2.2 Hz, 1 H), 5.73 (t, J=6.8 Hz, 1 H), 4.09 (s, 3 H), 3.78 (s, 3 H), 2.78 (t, J=7.6 Hz, 2 H), 2.35 (s, 3 H), 2.29 (s, 3 H), 2.17-2.14 (m, 2 H), 1.87 (quint, J=7.6 Hz, 2 H), 1.68-1.37 (m, 6 H), 1.35-1.19 (m, 6 H), 0.99-0.74 (m, 15 H); ¹³C NMR (100 MHz, CDCl₃) δ 179.4, 166.9, 153.8, 146.0, 140.4, 140.1, 134.4, 131.7, 126.8, 126.7, 63.0, 61.3, 29.2, 29.0, 27.3, 26.4, 25.9, 16.3, 13.7, 11.5, 9.94; FABMS (3-nitrobenzyl alcohol) m/z 654 (MH⁺).

N,2-Dimethoxy-3-methyl-5-[(1E)-5-(2-oxo-1,3-oxazolidin-3-yl)-1-(tributylstannanyl)pent-1-en-1-yl]benzenecarboximidoyl Chloride (56). The general hydrostannation procedure was followed using alkyne 50 (414 mg, 1.14 mmol), Pd(PPh₃)₄ (131 mg, 0.114 mmol) and n-Bu₃SnH (0.45 mL, 1.7 mmol) in THF (8 mL). The reaction mixture was stirred for 22 h and the product was purified by column chromatography on silica gel using 66% ethyl acetate-hexanes to afford the product 56 (503 mg, 67.2%) as a yellow oil: ¹H NMR (400 MHz, CDCl₃) δ 6.82 (d, J=2.2 Hz, 1 H), 6.80 (d, J=2.2 Hz, 1 H), 5.75 (t, J=7.1 Hz, 1 H), 4.23 (t, J=8.0 Hz, 2 H), 4.09 (s, 3 H), 3.78 (s, 3 H), 3.41 (t, J=8.0 Hz, 2 H), 3.19 (t, J=7.1 Hz, 2 H), 2.29 (s, 3 H), 2.09-2.04 (m, 2 H), 1.60 (quint, J=7.1 Hz, 2 H), 1.49-1.37 (m, 6 H), 1.33-1.19 (m, 6 H), 0.89-0.83 (m, 15 H); ¹³C NMR (100 MHz, CDCl₃) δ 158.4, 153.8, 145.4, 140.7, 140.2, 134.3, 131.8, 131.7, 126.7, 126.6, 63.0, 61.6, 61.3, 44.4, 43.8, 28.9, 27.5, 27.3, 27.1, 16.3, 13.7, 9.92; FABMS (3-nitrobenzyl alcohol) m/z 657 (MH⁺).

tert-Butyl (5E)-6-{4-Methoxy-3-methyl-5-[(methylsulfanyl)carbonyl]phenyl}-6-(tributylstannanyl)hex-5-enoate (65). The general hydrostannation procedure was followed using alkyne 64 (3.17 g, 8.75 mmol), Pd(PPh₃)₄ (1.01 g, 0.874 mmol) and n-Bu₃SnH (3.48 mL, 13.1 mmol) in THF (110 mL). The reaction mixture was stirred for 16 h and the product was purified by column chromatography on silica gel using 5% ethyl acetate-hexanes to afford the product 65 (5.47 g, 95.7%) as a colorless oil: ¹H NMR (270 MHz, CDCl₃) δ 7.11 (d, J=2.0 Hz, 1 H), 6.87 (d, J=2.0 Hz, 1 H), 5.73 (t, J=6.9 Hz, 1 H), 3.81 (s, 3 H), 2.44 (s, 3 H), 2.29 (s, 3 H), 2.16 (t, J=7.4 Hz, 2 H), 2.10-2.02 (m, 2 H), 1.64 (t, J=7.4 Hz, 2 H), 1.49-1.37 (m, 6 H), 1.39 (s, 9 H), 1.33-1.19 (m, 6 H), 0.89-0.81 (m, 15 H); ¹³C NMR (68 MHz, CDCl₃) δ 191.9, 172.6, 153.4, 144.4, 141.4, 140.2, 133.4, 131.8, 131.0, 125.1, 61.6, 34.9, 29.1, 28.8, 27.9, 27.2, 25.1, 16.0, 13.7, 12.3, 9.9.

General Procedure (Stille Coupling). A mixture of aryl iodide (1.00 equiv), organostannane (1.00 equiv) and cesium fluoride (3.00 equiv) in DMF was cooled to 0° C. and degassed for 10 min with argon. Then Pd(PPh₃)₄ (0.10 equiv) and CuI (1.20 equiv) were added to the mixture and the reaction mixture was stirred for 0.5 h at 60° C. under argon. After the reaction was complete, the reaction mixture was diluted with CH₂Cl₂ and water, shaken vigorously and filtered through celite with ethyl acetate. The organic layer was separated, washed with brine, dried over anhydrous Na₂SO₄, and concentrated. The product was purified by column chromatography on silica gel using ethyl acetate-hexanes.

5-[(1E)-1-(3,7-Dimethyl-2-oxo-2,3-dihydro-1,3-benzoxazol-5-yl)-5-(5-methyl-1,3,4-oxadiazol-2-yl)pent-1-en-1-yl]-N,2-dimethoxy-3-methylbenzenecarboximidoyl Fluoride (5). The general Stille coupling procedure was followed using aryl iodide 38 (189 mg, 0.654 mmol), organostannane 51 (416 mg, 0.654 mmol), CsF (300 mg, 1.97 mmol), Pd(PPh₃)₄ (76 mg, 0.065 mmol), and CuI (15 mg, 0.078 mmol) in dry DMF (12 mL). The reaction mixture was stirred for 17 h and the product was purified by column chromatography on silica gel using 20% ethyl acetate-hexanes to give the product 5 (93 mg, 28%) as a oil: ¹H NMR (270 MHz, CDCl₃) δ 7.20 (d, J=2.7 Hz, 1 H), 7.06 (d, J=2.7 Hz, 1 H), 6.74 (s, 1 H), 6.57 (s, 1 H), 5.98 (t, J=6.8 Hz, 1 H), 3.97 (s, 3 H), 3.87 (s, 3 H), 3.36 (d, J=6.8 Hz, 3 H), 2.82 (t, J=6.8 Hz, 2 H), 2.47 (s, 3 H), 2.33 (s, 3 H), 2.32 (s, 3 H), 2.28-2.19 (m, 2 H), 1.94 (quint, J=6.8 Hz, 2 H); ¹³C NMR (68 MHz, CDCl₃) δ 166.5, 156.1, 154.8, 149.3 (d, JCF=324.4 Hz), 141.1, 140.4, 138.3, 135.1 (d, J_{CCCF}=6.7 Hz), 132.6, 131.2, 128.8, 128.5, 123.5, 120.2, 119.8, 119.8, 104.5, 63.1 (d, J_{CONCF}=1.7 Hz), 61.0 (d, J_{COCCCF}=1.7 Hz), 29.7, 29.1, 28.3, 26.5, 24.8, 16.2, 14.5, 10.9; EIMS m/z (rel intensity) 508 (M⁺, 5.7), 457 (100), 371 (16.4), 359 (23.5), 111 (33.2), 98 (78.5); HRMS (EI) for C₂₇H₂₉FN405 calcd 508.2122 (M⁺), found 508.2116.

5-[(1E)-1-(3,7-Dimethyl-2-oxo-2,3-dihydro-1,3-benzoxazol-5-yl)-5-(2-oxo-1,3-oxazolidin-3-yl)pent-1-en-1-yl]-N,2-dimethoxy-3-methylbenzenecarboximidoyl Fluoride (6). The general Stille coupling procedure was followed using aryl iodide 38 (78 mg, 0.27 mmol), organostannane 53 (207 mg, 0.324 mmol), CsF (123 mg, 0.810 mmol), Pd(PPh₃)₄ (31 mg, 0.027 mmol) and CuI (62 mg, 0.32 mmol) in dry DMF (8 mL). The reaction mixture was stirred for 18 h and the product was purified by column chromatography on silica gel using 75% ethyl acetate-hexanes to give the product 6 (119 mg, 86.2%) as a clear oil: ¹H NMR (270 MHz, CDCl₃) δ 7.21 (s, 1 H), 7.06 (s, 1 H), 6.74 (s, 1 H), 6.61 (s, 1 H), 5.99 (t, J=7.5 Hz, 1 H), 4.27 (t, J=8.0 Hz, 2 H), 3.97 (s, 3 H), 3.87 (s, 3 H), 3.50 (t, J=8.0 Hz, 2 H), 3.35 (s, 3 H), 3.27 (t, J=7.5 Hz, 2 H), 2.33 (s, 3 H), 2.19 (s, 3 H), 2.19-2.11 (m, 2 H), 1.69 (quint, J=7.5 Hz, 2 H); ¹³C NMR (68 MHz, CDCl₃) δ 180.6, 162.2, 158.2, 156.1, 154.8, 149.3 (d, J_{CF}=324.5 Hz), 140.8, 140.4, 138.4, 135.2 (d, J_{CCCF}=6.2 Hz), 132.7, 131.2, 128.7, 128.3, 123.5, 120.0 (d, J_{CCF}=26.3 Hz), 104.5, 63.1 (d, J_{CONCF}=1.7 Hz), 61.6, 61.0 (d, J_{COCCCF}=1.7 Hz), 44.4, 43.7, 28.2, 27.5, 26.8, 15.5, 14.5; EIMS m/z (rel intensity) 511 (M⁺, 5.2), 460 (15), 347 (100), 100 (38), 56 (38); HRMS (EI) for C₂₇H₃₀FN₃O₆ calcd 511.2118 (M⁺), found 511.2113.

N,2-Dimethoxy-5-[(1Z)-1-(3-methoxy-7-methyl-1,2-benzoxazol-5-yl)-5-(5-methyl-1,3,4-oxadiazol-2-yl)pent-1-en-1-yl]-3-methylbenzenecarboximidoyl Fluoride (7). The general Stille coupling procedure was followed using aryl iodide 39 (107 mg, 0.370 mmol), organostannane 51 (235 mg, 0.370 mmol), CsF (168 mg, 1.11 mmol), Pd(PPh₃)₄ (42 mg, 0.037 mmol) and CuI (77 mg, 0.44 mmol) in dry DMF (15 mL). The reaction mixture was stirred for 18 h and the product was purified by column chromatography on silica gel using 20% ethyl acetate-hexanes to give the product 7 (165 mg, 87.8%) as a clear oil: ¹H NMR (270 MHz, CDCl₃) δ 7.39 (s, 1 H), 7.27 (s, 1 H), 7.17 (d, J=1.8 Hz, 1 H), 7.03 (d, J=1.8 Hz, 1 H), 6.01 (t, J=7.4 Hz, 1 H), 3.96 (s, 3 H), 3.87 (s, 3 H), 3.67 (s, 3 H), 2.81 (t, J=7.4 Hz, 2 H), 2.48 (s, 3 H), 2.36 (s, 3 H), 2.31 (s, 3 H), 2.27-2.19 (m, 2 H), 1.93 (quint, J=7.4 Hz, 2 H); ¹³C NMR (68 MHz, CDCl₃) δ 166.4, 163.4, 162.8, 157.9, 149.2 (d, J_{CF}=324.0 Hz), 140.4, 138.1, 135.1, 134.8, 132.7 (d, J_{CCCF}=6.7 Hz), 129.0, 128.5, 128.4, 120.1 (d, J_{CCF}=27.9 Hz), 119.8, 119.8, 115.7, 63.0 (d, J_{CONCF}=1.8 Hz), 60.9 (d, J_{COCCCF}=1.8 Hz), 32.6, 29.0, 26.4, 24.8, 16.2, 14.2, 10.9; EIMS m/z (rel intensity) 508 (M⁺, 21), 457 (52), 426 (100), 380 (41), 348 (21), 98 (37); HRMS (EI) for C₂₇H₂₉FN405 calcd 508.2122 (M⁺), found 508.2105.

N,2-Dimethoxy-5-[(1Z)-1-(3-methoxy-7-methyl-1,2-benzoxazol-5-yl)-5-(3-methyl-1,2,4-oxadiazol-5-yl)pent-1-en-1-yl]-3-methylbenzenecarboximidoyl Fluoride (8). The general Stille coupling procedure was followed using aryl iodide 39 (78 mg, 0.27 mmol), organostannane 52 (207 mg, 0.324 mmol), CsF (123 mg, 0.810 mmol), Pd(PPh₃)₄ (31 mg, 0.027 mmol) and CuI (6 mg, 0.027 mmol) in dry DMF (8 mL). The reaction mixture was stirred for 16 h and was purified by column chromatography on silica gel using 50% ethyl acetate-hexanes to give the product 8 (48 mg, 35%) as a oil: ¹H NMR (270 MHz, CDCl₃) δ 7.38 (d, J=1.2 Hz, 1 H), 7.27 (d, J=1.2 Hz, 1 H), 7.18 (d, J=1.8 Hz, 1 H), 7.02 (d, J=1.8 Hz, 1 H), 6.00 (t, J=7.4 Hz, 1 H), 3.96 (s, 3 H), 3.87 (s, 3 H), 3.67 (s, 3 H), 2.85 (t, J=7.4 Hz, 2 H), 2.35 (s, 3 H), 2.35 (s, 3 H), 2.31 (s, 3 H), 2.27-2.19 (m, 2 H), 1.96 (quint, J=7.4 Hz, 2 H); ¹³C NMR (8 MHz, CDCl₃) δ 178.9, 166.8, 162.8, 158.0, 156.2, 149.3 (d, J_{CF}=324.4 Hz), 140.6, 138.1, 135.1, 134.8, 132.8, 132.8, 128.9, 128.6, 128.5, 119.9, 119.8, 115.8, 63.1 (d, J_{CONCF}=2.2 Hz), 61.0 (d, J_{COCCCF}=2.2 Hz), 32.7, 29.0, 26.5, 25.9, 16.2, 14.2, 11.6; EIMS m/z (rel intensity) 508 (M⁺, 4.3), 457 (60), 426 (100), 380 (41), 348 (20); HRMS (EI) for C₂₇H₂₉FN₄O₅ calcd 508.2122 (M⁺), found 508.2145.

N,2-Dimethoxy-5-[(1Z)-1-(3-methoxy-7-methyl-1,2-benzoxazol-5-yl)-5-(2-oxo-1,3- oxazolidin-3-yl)pent-1-en-1-yl]-3-methylbenzenecarboximidoyl Fluoride (9). The general Stille coupling procedure was followed using aryl iodide 39 (135 mg, 0.469 mmol), organostannane 53 (300 mg, 0.469 mmol), CsF (214 mg, 1.41 mmol), Pd(PPh₃)₄ (54 mg, 0.047 mmol) and CuI (107 mg, 0.563 mmol) in dry DMF (10 mL). The reaction mixture was stirred for 16 h and purified by column chromatography on silica gel using 75% ethyl acetate-hexanes to give the product 9 (202 mg, 82.2%) as a clear oil: ¹H NMR (270 MHz, CDCl₃) δ 7.31 (s, 1 H), 7.24 (s, 1 H), 7.12 (s, 1 H), 7.00 (s, 1 H), 5.97 (t, J=7.5 Hz, 1 H), 4.20 (t, J=8.0 Hz, 2 H), 3.89 (s, 3 H), 3.82 (s, 3 H), 3.60 (s, 3 H), 3.43 (t, J=8.0 Hz, 2 H), 3.20 (t, J=7.5 Hz, 2 H), 2.29 (s, 3 H), 2.26 (s, 3 H), 2.12-2.04 (m, 2 H), 1.63 (quint, J=7.5 Hz, 2 H); ¹³C NMR (68 MHz, CDCl₃) δ 162.8, 158.2, 157.9, 156.1, 149.2 (d, J_{CF}=324.5 Hz), 140.1, 138.1, 135.2, 134.9, 132.8, 132.8, 129.1, 128.4 (d, J_{CCCF}=4.5 Hz), 120.0 (d, J_{CCF}=27.4 Hz), 119.8, 119.8, 115.6, 63.0 (d, J_{CONCF}=2.2 Hz), 61.6, 61.0 (d, J_{COCCCF}=2.2 Hz), 44.4, 43.7, 32.6, 27.4, 26.8, 16.1, 14.1; EIMS m/z (rel intensity) 511 (M⁺, 17), 460 (23), 429 (39), 380 (33), 373 (58), 342 (71), 316 (29), 100 (100), 56 (76); HRMS (EI) for C₂₇H₃₀FN₃O₆ calcd 511.2119 (M⁺), found 511.2104.

5-[(1Z)-1-(3-Cyanophenyl)-5-(5-methyl-1,3,4-oxadiazol-2-yl)pent-1-en-1-yl]-N,2-dimethoxy-3-methylbenzenecarboximidoyl Fluoride (10). The general Stille coupling procedure was followed using 3-iodobenzonitrile 40 (90 mg, 0.39 mmol), organostannane 51 (250 mg, 0.393 mmol), CsF (179 mg, 1.18 mmol), Pd(PPh₃)₄ (45 mg, 0.039 mmol) and CuI (90 mg, 0.472 mmol) in dry DMF (10 mL). The reaction mixture was stirred for 17 h and purified by column chromatography on silica gel using 50% ethyl acetate-hexanes to give the product 10 (141 mg, 80.3%) as a clear oil: ¹H NMR (270 MHz, CDCl₃) δ 7.52 (dd, J=5.7, 1.8 Hz, 1 H), 7.46-7.35 (m, 3 H), 7.17 (d, J=1.9 Hz, 1 H), 7.03 (d, J=1.9 Hz, 1 H), 6.10 (t, J=7.4 Hz, 1 H), 3.97 (s, 3 H), 3.88 (s, 3 H), 2.81 (t, J=7.4 Hz, 2 H), 2.48 (s, 3 H), 2.33 (s, 3 H), 2.28-2.20 (m, 2 H), 1.94 (quint, J=7.4 Hz, 2 H); ¹³C NMR (68 MHz, CDCl₃) δ 166.2, 163.3, 156.3, 149.0 (d, J_{CF}=324.5 Hz), 142.8, 139.5, 135.0, 133.8, 132.9, 131.0, 130.8, 130.4, 130.3, 128.8, 128.3 (d, J_{CCCF}=4.5 Hz), 120.2 (d, J_{CCF}=27.4 Hz), 118.5, 112.1, 63.0 (d, J_{CONCF}=2.0 Hz), 60.9 (d, J_{COCCCF}=2.0 Hz), 29.0, 26.2, 24.7, 16.1, 10.8; EIMS m/z (rel intensity) 448 (M⁺, 13), 397 (100), 351 (29), 299 (26), 98 (61); HRMS (EI) for C₂₅H₂₅FN₄O₃ calcd 448.1911 (M⁺), found 448.1894.

5-[(1Z)-1-(3-Cyanophenyl)-5-(3-methyl-1,2,4-oxadiazol-5-yl)pent-1-en-1-yl]-N,2-dimethoxy-3-methylbenzenecarboximidoyl Fluoride (11). The general Stille coupling procedure was followed using 3-iodobenzonitrile 40 (69 mg, 0.30 mmol), organostannane 52 (242 mg, 0.362 mmol), CsF (137 mg, 0.903 mmol), Pd(PPh₃)₄ (34 mg, 0.030 mmol), and CuI (7 mg, 0.03 mmol) in dry DMF (8 mL). The reaction mixture was stirred for 16 h and was purified by column chromatography on silica gel using 20% ethyl acetate-hexanes to give the product 11 (72 mg, 54%) as a oil: ¹H NMR (270 MHz, CDCl₃) δ 7.54-7.40 (m, 4 H), 7.17 (d, J=1.8 Hz, 1 H), 7.02 (d, J=1.8 Hz, 1 H), 6.09 (t, J=7.4 Hz, 1 H), 3.97 (s, 3 H), 3.88 (s, 3 H), 2.85 (t, J=7.4 Hz, 2 H), 2.35 (s, 3 H), 2.33 (s, 3 H), 2.29-2.23 (m, 2 H), 1.97 (quint, J=7.4 Hz, 2 H); ¹³C NMR (68 MHz, CDCl₃) δ 178.7, 166.8, 156.4, 149.2 (d, J_{CF}=324.2 Hz), 142.8, 139.8, 135.0, 134.2, 133.8, 133.0, 131.1, 130.7, 130.6, 128.5 (d, J_{CCCF}=4.5 Hz), 128.0, 120.4 (d, J_{CCF}=27.4 Hz), 118.6, 112.2, 63.1 (d, J_{CONCF}=2.0 Hz), 61.0 (d, J_{COCCCF}=2.0 Hz), 29.0, 26.3, 25.9, 16.2, 11.5; EIMS m/z (rel intensity) 448 (M⁺, 9.7), 397 (100), 299 (42), 262 (69), 224 (34), 183 (40); HRMS (EI) for C₂₅H25FN403 calcd 448.1911 (M⁺), found 448.1910.

5-[(1Z)-1-(3-Cyanophenyl)-5-(2-oxo-1,3-oxazolidin-3-yl)pent-1-en-1-yl]-N,2-dimethoxy-3-methylbenzenecarboximidoyl Fluoride (12). The general Stille coupling procedure was followed using 3-iodobenzonitrile 40 (107 mg, 0.469 mmol), organostannane 53 (300 mg, 0.469 mmol), CsF (214 mg, 1.41 mmol), Pd(PPh₃)₄ (54 mg, 0.047 mmol) and CuI (107 mg, 0.563 mmol) in dry DMF (10 mL). The reaction mixture was stirred for 16 h and purified by column chromatography on silica gel using 60% ethyl acetate-hexanes to give the product 12 (166 mg, 78.6%) as a clear oil: ¹H NMR (270 MHz, CDCl₃) δ 7.53-7.36 (m, 4 H), 7.18 (d, J=2.2 Hz, 1 H), 7.05 (d, J=2.2 Hz, 1 H), 6.13 (t, J=7.4 Hz, 1 H), 4.27 (t, J=8.0 Hz, 2 H), 3.97 (s, 3 H), 3.88 (s, 3 H), 3.49 (t, J=8.0 Hz, 2 H), 3.27 (t, J=7.4 Hz, 2 H), 2.34 (s, 3 H), 2.20-2.12 (m, 2 H), 1.71 (quint, J=7.7 Hz, 2 H); ¹³C NMR (68 MHz, CDCl₃) δ 158.1, 156.3, 149.0 (d, J_{CF}=324.5 Hz), 142.8, 139.2, 135.0, 133.9, 133.0, 131.1, 130.9, 130.4, 130.3, 128.8, 128.3 (d, J_{CCCF}=3.9 Hz), 120.2 (d, J_{CCF}=27.4 Hz), 118.6, 112.1, 63.0 (d, J_{CONCF}=1.9 Hz), 61.5, 60.9 (d, J_{COCCCF}=1.9 Hz), 44.3, 43.5, 27.1, 26.8, 16.1; EIMS m/z (rel intensity) 451 (M⁺, 83), 400 (21), 313 (100), 287 (25), 100 (87), 56 (26); HRMS (EI) for C₂₅H₂₆FN₃O₄ calcd 451.1907 (M⁺), found 451.1915.

5-[(1Z)-1-(4-Cyanophenyl)-5-(5-methyl-1,3,4-oxadiazol-2-yl)pent-1-en-1-yl]-N,2-dimethoxy-3-methylbenzenecarboximidoyl Fluoride (13). The general Stille coupling procedure was followed using 4-iodobenzonitrile 41 (70 mg, 0.31 mmol), organostannane 51 (240 mg, 0.371 mmol), CsF (140 mg, 0.942 mmol), Pd(PPh₃)₄ (30 mg, 0.031 mmol) and CuI (70 mg, 0.38 mmol) in dry DMF (10 mL). The reaction mixture was stirred for 17 h and the product was purified by column chromatography on silica gel using 50% ethyl acetate-hexanes to give the product 13 (68 mg, 49%) as a clear oil: ¹H NMR (270 MHz, CDCl₃) δ 7.56 (d, J=7.7 Hz, 2 H), 7.28 (d, J=7.7 Hz, 2 H), 7.18 (s, 1 H), 7.04 (s, 1 H), 6.18 (t, J=7.4 Hz, 1 H), 3.96 (s, 3 H), 3.87 (s, 3 H), 2.81 (t, J=7.4 Hz, 2 H), 2.47 (s, 3 H), 2.32 (s, 3 H), 2.30-2.22 (m, 2 H), 1.95 (quint, J=7.4 Hz, 2 H); ¹³C NMR (68 MHz, CDCl₃) δ 166.1, 163.3, 156.2, 149.0 (d, J_{CF}=324.5 Hz), 145.9, 140.0, 135.0, 133.7, 132.8, 131.7, 131.7, 128.3 (d, J_{CCCF}=4.5 Hz), 127.3, 120.1 (d, J_{CCF}=27.4 Hz), 118.6, 110.3, 62.9 (d, J_{CONCF}=2.2 Hz), 60.8 (d, J_{COCCCF}=2.2 Hz), 29.0, 26.1, 24.6, 16.0, 10.7; EIMS m/z (rel intensity) 448 (M⁺, 12), 397 (100), 351 (20), 299 (27), 98 (71); HRMS (EI) for C₂₅H25FN403 calcd 448.1911 (M⁺), found 448.1897.

5-[(1Z)-1-(4-Cyanophenyl)-5-(3-methyl-1,2,4-oxadiazol-5-yl)pent-1-en-1-yl]-N,2-dimethoxy-3-methylbenzenecarboximidoyl Fluoride (14). The general Stille coupling procedure was followed using 4-iodobenzonitrile 41 (69 mg, 0.30 mmol), organostannane 52 (242 mg, 0.362 mmol), CsF (137 mg, 0.901 mmol), Pd(PPh₃)₄ (34 mg, 0.030 mmol) and CuI (7 mg, 0.03 mmol) in dry DMF (8 mL). The reaction mixture was stirred for 18 h and the product was purified by column chromatography on silica gel using 20% ethyl acetate-hexanes to give the product 14 (94 mg, 70%) as an oil: ¹H NMR (270 MHz, CDCl₃) δ 7.48 (d, J=8.7 Hz, 2 H), 7.20 (d, J=8.7 Hz, 2 H), 7.10 (d, J=1.8 Hz, 1 H), 6.95 (d, J=1.8 Hz, 1 H), 6.09 (t, J=7.4 Hz, 1 H), 3.89 (s, 3 H), 3.79 (s, 3 H), 2.78 (t, J=7.4 Hz, 2 H), 2.27 (s, 3 H), 2.24 (s, 3 H) 2.29-2.21 (m, 2 H), 1.90 (quint, J=7.4 Hz, 2 H); ¹³C NMR (68 MHz, CDCl₃) δ 178.7, 166.7, 156.3, 149.1 (d, J_{CF}=324.2 Hz), 145.9, 140.2, 135.0, 133.8, 132.9, 131.8, 131.5, 128.5 (d, J_{CCCF}=4.5Hz), 127.4, 120.3 (d, J_{CCF}=27.4 Hz), 118.6, 110.5, 63.0 (d, J_{CONCF}=2.2 Hz), 60.9 (d, J_{COCCCF}=2.2 Hz), 29.0, 26.2, 25.8, 16.1, 11.4; EIMS m/z (rel intensity) 448 (M⁺, 8.7), 397 (100), 299 (42), 111 (16), 98 (22); HRMS (EI) for C₂₅H₂₅FN₄O₃ calcd 448.1911 (M⁺), found 448.1910.

5-[(1Z)-1-(4-Cyanophenyl)-5-(2-oxo-1,3-oxazolidin-3-yl)pent-1-en-1-yl]-N,2-dimethoxy-3-methylbenzenecarboximidoyl Fluoride (15). The general Stille coupling procedure was followed using 4-iodobenzonitrile 41 (107 mg, 0.469 mmol), organostannane 53 (300 mg, 0.469 mmol), CsF (214 mg, 1.41 mmol), Pd(PPh₃)₄ (54 mg, 0.047 mmol) and CuI (107 mg, 0.563 mmol) in dry DMF (10 mL). The reaction mixture was stirred for 16 h and purified by column chromatography on silica gel using 60% ethyl acetate-hexanes to give the product 15 (150 mg, 70.8%) as a clear oil: ¹H NMR (270 MHz, CDCl₃) δ 7.55 (d, J=8.2 Hz, 2 H), 7.29 (d, J=8.2 Hz, 2 H), 7.18 (s, 1 H), 7.05 (s, 1 H), 6.21 (t, J=7.4 Hz, 1 H), 4.26 (t, J=8.0 Hz, 2 H), 3.96 (s, 3 H), 3.87 (s, 3 H), 3.49 (t, J=8.0 Hz, 2 H), 3.27 (t, J=7.4 Hz, 2 H), 2.33 (s, 3 H), 2.21-2.12 (m, 2 H), 1.71 (quint, J=7.4 Hz, 2 H); ¹³C NMR (68 MHz, CDCl₃) δ 158.2, 156.4, 149.1 (d, J_{CF}=324.5Hz), 146.0, 139.9, 135.2, 134.0, 133.0, 131.9, 131.9, 128.4 (d, J_{CCCF}=3.9 Hz), 127.5, 120.3 (d, J_{CCF}=27.9 Hz), 118.8, 110.4, 63.1 (d, J_{CONCF}=2.2 Hz), 61.6, 61.0 (d, J_{COCCCF}=2.2 Hz), 44.4, 43.6, 27.2, 27.0, 16.2; EIMS m/z (rel intensity) 451 (M⁺, 12), 400 (15), 313 (100), 287 (23), 100 (66); HRMS (EI) for C₂₅H₂₆FN₃O₄ calcd 451.1907 (M⁺), found 451.1903.

5-[(1E)-1-(3,7-Dimethyl-2-oxo-2,3-dihydro-1,3-benzoxazol-5-yl)-5-(5-methyl-1,3,4-oxadiazol-2-yl)pent-1-en-1-yl]-N,2-dimethoxy-3-methylbenzenecarboximidoyl Chloride (16). The general Stille coupling procedure was followed using aryl iodide 38 (74 mg, 0.26 mmol), organostannane 54 (200 mg, 0.307 mmol), CsF (120 mg, 0.768 mmol), Pd(PPh₃)₄ (30 mg, 0.026 mmol) and CuI (6 mg, 0.03 mmol) in dry DMF (10 mL). The reaction mixture was stirred for 17 h and the product was purified by column chromatography on silica gel using 50% ethyl acetate-hexanes to give the product 16 (111 mg, 82.4%) as a clear oil: ¹H NMR (270 MHz, CDCl₃) δ 7.09 (d, J=2.0 Hz, 1 H), 7.02 (s, J=2.0 Hz, 1 H), 6.76 (s, 1 H), 6.59 (s, 1 H), 5.96 (t, J=7.4 Hz, 1 H), 4.10 (s, 3 H), 3.85 (s, 3 H), 3.35 (s, 3 H), 2.82 (t, J=7.5 Hz, 2 H), 2.48 (s, 3 H), 2.33 (s, 3 H), 2.32 (s, 3 H), 2.3.-2.22 (m, 2 H), 1.95 (quint, J=7.5 Hz, 2 H); ¹³C NMR (68 MHz, CDCl₃) δ 166.4, 163.3, 155.4, 154.7, 141.1, 140.3, 138.4, 134.8, 134.3, 133.9, 132.0, 131.1, 129.4, 128.8, 126.8, 123.5, 119.7, 104.5, 62.9, 61.2, 29.0, 28.2, 26.5, 24.8, 16.3, 14.4, 10.9; EIMS m/z (rel intensity) 524 (M⁺, 2.9), 526 (M⁺+2, 1.1), 457 (64), 359 (20), 345 (16), 111 (20), 98 (100); HRMS (EI) for C₂₅H25³⁵ClN₄O₃ calcd 524.1827 (M⁺), found 524.1813, C₂₅H₂₅³⁷ClN₄O₃ calcd 526.1797 (M⁺+2), found 526.1768.

5-[(1E)-1-(3,7-Dimethyl-2-oxo-2,3-dihydro-1,3-benzoxazol-5-yl)-5-(3-methyl-1,2,4-oxadiazol-5-yl)pent-1-en-1-yl]-N,2-dimethoxy-3-methylbenzenecarboximidoyl Chloride (17). The general Stille coupling procedure was followed using aryl iodide 38 (89 mg, 0.31 mmol), organostannane 55 (240 mg, 0.370 mmol), CsF (140 mg, 0.924 mmol), Pd(PPh₃)₄ (35 mg, 0.031 mmol) and CuI (70 mg, 0.37 mmol) in dry DMF (8 mL). The reaction mixture was stirred for 18 h and the product was purified by column chromatography on silica gel using 20% ethyl acetate-hexanes to give the product 17 (119 mg, 73.6%) as an oil: ¹H NMR (270 MHz, CDCl₃) δ 7.10 (d, J=1.8 Hz, 1 H), 7.01 (d, J=1.8 Hz, 1 H), 6.77 (d, J=1.2 Hz, 1 H), 6.59 (d, J=1.2 Hz, 1 H), 5.95 (t, J=7.4 Hz, 1 H), 4.10 (s, 3 H), 3.85(s, 3 H), 3.35 (s, 3 H), 2.87 (t, J=7.6 Hz, 2 H), 2.36 (s, 3 H), 2.33 (s, 3 H), 2.32 (s, 3 H), 2.23 (m, 2 H), 1.98 (quint, J=7.4 Hz, 2 H); ¹³C NMR (68 MHz, CDCl₃) δ 178.9, 166.7, 155.4, 154.7, 141.2, 140.3, 138.4, 134.8, 134.3, 133.9, 132.0, 131.1, 129.4, 128.6, 126.8, 123.5, 119.7, 104.5, 63.0, 61.2, 29.0, 28.2, 26.5, 25.9, 16.4, 14.5, 11.5; EIMS m/z (rel intensity) 524 (M⁺, 4.9), 526 (M⁺+2, 1.8), 457 (100), 359 (21), 347 (19), 111 (17), 98 (18); HRMS (EI) for C₂₇t129³⁵ClN₄O₅ calcd 524.1826 (M⁺), found 524.1808, C₂₇H₂₉³⁷ClN₄O₅ calcd 526.1797 (M⁺+2), found 526.1780.

5-[(1E)-1-(3,7-Dimethyl-2-oxo-2,3-dihydro-1,3-benzoxazol-5-yl)-5-(2-oxo-1,3-oxazolidin-3-yl)pent-1-en-1-yl]-N,2-dimethoxy-3-methylbenzenecarboximidoyl Chloride (18). The general Stille coupling procedure was followed using aryl iodide 38 (167 mg, 0.578 mmol), organostannane 56 (454 mg, 0.694 mmol), CsF (260 mg, 1.71 mmol), Pd(PPh₃)₄ (67 mg, 0.058 mmol) and CuI (130 mg, 0.694 mmol) in dry DMF (20 mL). The reaction mixture was stirred for 23 h and the product was purified by column chromatography on silica gel using 75% ethyl acetate-hexanes to give the product 18 (209 mg, 68.3%) as a clear oil: ¹H NMR (400 MHz, CDCl₃) δ 7.09 (d, J=2.0 Hz, 1 H), 7.02 (d, J=2.0 Hz, 1 H), 6.76 (s, 1 H), 6.61 (s, 1 H), 5.98 (t, J=7.4 Hz, 1 H), 4.26 (t, J=8.0 Hz, 2 H), 4.09 (s, 3 H), 3.85 (s, 3 H), 3.48 (t, J=8.0 Hz, 2 H), 3.35 (s, 3 H), 3.27 (t, J=7.4 Hz, 2 H), 2.33 (s, 3 H), 2.32 (s, 3 H), 2.19-2.13 (m, 2 H), 1.70 (quint, J=7.5 Hz, 2 H); ¹³C NMR (100 MHz, CDCl₃) δ 158.4, 155.7, 155.1, 141.1, 140.6, 138.6, 135.1, 134.7, 134.2, 132.3, 131.3, 129.6, 129.0, 127.0, 123.7, 120.0, 104.7, 63.1, 61.6, 61.3, 44.5, 43.7, 28.3, 27.5, 26.8, 16.4, 14.5; EIMS m/z (rel intensity) 527 (M⁺, 3.5), 529 (M⁺+2, 1.3), 460 (20), 387 (21), 373 (51), 347 (100), 321 (20), 100 (26); HRMS (EI) for C₂₇H₃₀³⁵ClN₃O₆ calcd 527.1823 (M⁺), found 527.1813, C₂₇H₃₀³⁷ClN₃O₆ calcd 529.1794 (M⁺+2), found 529.1800.

N,2-Dimethoxy-5-[(1Z)-1-(3-methoxy-7-methyl-1,2-benzoxazol-5-yl)-5-(5-methyl-1,3,4-oxadiazol-2-yl)pent-1-en-1-yl]-3-methylbenzenecarboximidoyl Chloride (19). The general Stille coupling procedure was followed using aryl iodide 39 (50 mg, 0.17 mmol), organostannane 54 (135 mg, 0.208 mmol), CsF (78 mg, 0.52 mmol), Pd(PPh₃)₄ (20 mg, 0.017 mmol) and CuI (36 mg, 0.21 mmol) in dry DMF (8 mL). The reaction mixture was stirred for 18 h and the product was purified by column chromatography on silica gel using 15% ethyl acetate-hexanes to give the product 19 (79 mg, 88%) as a clear oil: ¹H NMR (270 MHz, CDCl₃) δ 7.41 (d, J=1.2 Hz, 1 H), 7.29 (d, J=1.2 Hz, 1 H), 7.07 (d, J=2.2 Hz, 1 H), 7.00 (d, J=2.2 Hz, 1 H), 6.00 (t, J=7.4 Hz, 1 H), 4.09 (s, 3 H), 3.86 (s, 3 H), 3.67 (s, 3 H), 2.81 (t, J=7.4, 2 H), 2.48 (s, 3 H), 2.36 (s, 3 H), 2.31 (s, 3 H), 2.30-2.21 (m, 2 H), 1.93 (quint, J=7.4 Hz, 2 H); ¹³C NMR (68 MHz, CDCl₃) δ 166.4, 163.3, 162.7, 157.8, 155.5, 140.5, 138.2, 134.6, 134.3, 133.8, 132.9, 132.1, 129.4, 129.0, 126.9, 119.8, 119.7, 115.6, 62.9, 61.2, 32.6, 29.0, 26.4, 24.7, 16.3, 14.1, 10.9: EIMS m/z (rel intensity) 524 (M⁺, 5.3), 526 (M⁺+2, 2.0), 457 (50), 426 (100), 330 (36), 98 (69); HRMS (EI) for C₂₇H₂₉³⁵ClN₄O₅ calcd 524.1826 (M⁺), found 524.1807, C₂₇H₂₉³⁷ClN₄O₅ calcd 526.1797 (M⁺+2), found 524.1804.

N,2-Dimethoxy-5-[(1Z)-1-(3-methoxy-7-methyl-1,2-benzoxazol-5-yl)-5-(3-methyl-1,2,4-oxadiazol-5-yl)pent-1-en-1-yl]-3-methylbenzenecarboximidoyl Chloride (20). The general Stille coupling procedure was followed using aryl iodide 39 (85 mg, 0.29 mmol), organostannane 55 (230 mg, 0.353 mmol), CsF (134 mg, 0.882 mmol), Pd(PPh₃)₄ (34 mg, 0.029 mmol) and CuI (7 mg, 0.03 mmol) in dry DMF (8 mL). The reaction mixture was stirred for 18 h and the product was purified by column chromatography on silica gel using 50% ethyl acetate-hexanes to give the product 20 (46 mg, 30%) as a oil: ¹H NMR (270 MHz, CDCl₃) δ 7.40 (s, 1 H), 7.27 (s, 1 H), 7.07 (d, J=1.8 Hz, 1 H), 6.98 (d, J=1.8 Hz, 1 H), 5.99 (t, J=7.4 Hz, 1 H), 4.09 (s, 3 H), 3.87 (s, 3 H), 3.67 (s, 3 H), 2.85 (t, J=7.4 Hz, 2 H), 2.36 (s, 3 H), 2.36 (s, 3 H), 2.31 (s, 3 H), 2.25-2.17 (m, 2 H), 1.97 (quint, J=7.4 Hz, 2 H); ¹³C NMR (68 MHz, CDCl₃) δ 178.8, 166.6, 162.8, 157.9, 155.5, 140.6, 138.2, 134.5, 134.2, 133.9, 132.8, 132.1, 129.4, 128.8, 126.9, 119.9, 119.7, 115.7, 62.9, 61.2, 32.6, 29.0, 26.5, 25.8, 16.3, 14.1, 11.5; EIMS m/z (rel intensity) 524 (M⁺, 3.1), 526 (M⁺+2, 1.4), 457 (87), 426 (100), 396 (18), 359 (18), 330 (34), 329 (24), 98 (19); HRMS (EI) for C₂₇H₂₉³⁵ClN₄O₅ calcd 524.1826 (M⁺), found 524.1800, C₂₇t1₂₉³⁷ClN₄O₅ calcd 526.1797 (M⁺+2), found 526.1814.

5-[(1Z)-1-(3-Cyanophenyl)-5-(5-methyl-1,3,4-oxadiazol-2-yl)pent-1-en-1-yl]-N,2-dimethoxy-3-methylbenzenecarboximidoyl Chloride (21). The general Stille coupling procedure was followed using 3-iodobenzonitrile 40 (35 mg, 0.15 mmol), organostannane 54 (120 mg, 0.184 mmol), CsF (69 mg, 0.46 mmol), Pd(PPh₃)₄ (17 mg, 0.015 mmol) and CuI (34 mg, 0.18 mmol) in dry DMF (8 mL). The reaction mixture was stirred for 21 h and the product was purified by column chromatography on silica gel using 15% ethyl acetate-hexanes to give the product 21 (64 mg, 90%) as a oil: ¹H NMR (270 MHz, CDCl₃) δ 7.54-7.41 (m, 4 H), 7.06 (s, 1 H), 7.00 (s, 1 H), 6.10 (t, J=7.4 Hz, 1 H), 4.10 (s, 3 H), 3.86 (s, 3 H), 2.82 (t, J=7.4 Hz, 2 H), 2.48 (s, 3 H), 2.33 (s, 3 H), 2.30-2.23 (m, 2 H), 1.95 (quint, J=7.4 Hz, 2 H); ¹³C NMR (68 MHz, CDCl₃) δ 166.3, 163.5, 155.8, 143.0, 139.8, 134.3, 133.8, 133.7, 132.5, 131.2, 130.8, 130.6, 130.5, 129.4, 128.9, 127.2, 118.7, 112.3, 63.0, 61.3, 29.1, 26.4, 24.8, 16.4, 11.0; EIMS m/z (rel intensity) 464 (M⁺, 2.0), 466 (M⁺+2, 0.7), 398 (17), 397 (51), 299 (13), 98 (100); HRMS (EI) for C₂₅H25³⁵ClN₄O₃ calcd 464.1615 (M⁺), found 464.1600, C₂₅H₂₅³⁷ClN₄O₃ calcd 466.1586 (M⁺+2), found 466.1584.

5-[(1Z)-1-(3-Cyanophenyl)-5-(3-methyl-1,2,4-oxadiazol-5-yl)pent-1-en-1-yl]-N,2-dimethoxy-3-methylbenzenecarboximidoyl Chloride (22). The general Stille coupling procedure was followed using 3-iodobenzonitrile 40 (70 mg, 0.31 mmol), organostannane 55 (240 mg, 0.37 mmol), CsF (140 mg, 0.918 mmol), Pd(PPh₃)₄ (35 mg, 0.031 mmol) and CuI (70 mg, 0.367 mmol) in dry DMF (8 mL). The reaction mixture was stirred for 18 h and the product was purified by column chromatography on silica gel using 20% ethyl acetate-hexanes to give the product 22 (82 mg, 58%) as a oil: ¹H NMR (400 MHz, CDCl₃) δ 7.58-7.27 (m, 4 H), 7.06 (d, J=2.2 Hz, 1 H), 6.98 (d, J=2.2 Hz, 1 H), 6.08 (t, J=7.5 Hz, 1 H), 4.10 (s, 3 H), 3.86 (s, 3 H), 2.86 (t, J=7.5 Hz, 2 H), 2.36 (s, 3 H), 2.33 (s, 3 H), 2.30-2.24 (m, 2 H), 1.98 (quint, J=7.5 Hz, 2 H); ¹³C NMR (68 MHz, CDCl₃) δ 179.0, 167.0, 156.0, 143.2, 140.2, 134.4, 134.0, 133.8, 132.7, 131.4, 130.8, 130.8, 130.6, 129.6, 129.1, 127.4, 118.8, 112.4, 63.1, 61.4, 29.1, 26.3, 25.9, 16.4, 11.5; EIMS m/z (rel intensity) 464 (M⁺, 7.4), 466 (M⁺+2, 2.9), 398 (28), 397 (100), 299 (34), 98 (39); HRMS (EI) for C₂₅H25³⁵ClN₄O₃ calcd 464.1615 (M⁺), found 464.1591, C₂₅H₂₅³⁷ClN₄O₃ calcd 466.1586 (M⁺+2), found 466.1572.

5-[(1Z)-1-(4-Cyanophenyl)-5-(5-methyl-1,3,4-oxadiazol-2-yl)pent-1-en-1-yl]-N,2-dimethoxy-3-methylbenzenecarboximidoyl Chloride (23). The general Stille coupling procedure was followed using 4-iodobenzonitrile 41 (44 mg, 0.19 mmol), organostannane 54 (150 mg, 0.230 mmol), CsF (88 mg, 0.58 mmol), Pd(PPh₃)₄ (22 mg, 0.019 mmol) and CuI (44 mg, 0.23 mmol) in dry DMF (8 mL). The reaction mixture was stirred for 16 h and the product was purified by column chromatography on silica gel using 15% ethyl acetate-hexanes to give the product 23 (73 mg, 83%) as an oil: ¹H NMR (270 MHz, CDCl₃) δ 7.56 (d, J=8.6 Hz, 2 H), 7.30 (d, J=8.6 Hz, 2 H), 7.06 (d, J=2.3 Hz, 1 H), 7.00 (d, J=2.3 Hz, 1 H), 6.16 (t, J=7.5 Hz, 1 H), 4.09 (s, 3 H), 3.85 (s, 3 H), 2.81 (t, J=7.5 Hz, 2 H), 2.47 (s, 3 H), 2.32 (s, 3 H), 2.32-2.23 (m, 2 H), 1.95 (quint, J=7.5 Hz, 2 H); ¹³C NMR (68 MHz, CDCl₃) δ 166.2, 163.4, 155.7, 146.1, 140.2, 134.3, 133.8, 133.6, 132.4, 131.8, 131.6, 129.4, 127.5, 127.1, 118.7, 110.4, 63.0, 61.2, 29.1, 26.2, 24.8, 16.3, 10.9; EIMS m/z (rel intensity) 464 (M⁺, 1.1), 466 (M⁺+2, 0.4), 398 (11), 397 (28), 299 (10), 98 (100); HRMS (EI) for C₂₅H₂₅³⁵ClN₄O₃ calcd 464.1616 (M⁺), found 464.1608, C₂₅H₂₅³⁷ClN₄O₃ calcd 466.1586 (M⁺+2), found 466.1583.

5-[(1Z)-1-(4-Cyanophenyl)-5-(3-methyl-1,2,4-oxadiazol-5-yl)pent-1-en-1-yl]-N,2-dimethoxy-3-methylbenzenecarboximidoyl Chloride (24). The general Stille coupling procedure was followed using 4-iodobenzonitrile 41 (70 mg, 0.31 mmol), organostannane 55 (240 mg, 0.367 mmol), CsF (140 mg, 0.918 mmol), Pd(PPh₃)₄ (35 mg, 0.031 mmol) and CuI (70 mg, 0.37 mmol) in dry DMF (8 mL). The reaction mixture was stirred for 18 h and the product was purified by column chromatography on silica gel using 20% ethyl acetate-hexanes to give the product 24 (106 mg, 74.3%) as an oil: ¹H NMR (400 MHz, CDCl₃) δ 7.56 (d, J=8.4 Hz, 2 H), 7.29 (d, J=8.4 Hz, 2 H), 7.06 (d, J=2.4 Hz, 1 H), 6.99 (d, J=2.4 Hz, 1 H), 6.16 (t, J=7.6 Hz, 1 H), 4.09 (s, 3 H), 3.86 (s, 3 H), 2.86 (t, J=7.6 Hz, 2 H), 2.35 (s, 3 H), 2.32 (s, 3 H), 2.30−2.25 (m, 2 H), 1.98 (quint, J=7.6 Hz, 2 H); ¹³C NMR (100 MHz, CDCl₃) δ 179.0, 167.0, 156.0, 146.2, 140.6, 134.4, 134.0, 133.78, 132.6, 132.0, 131.6, 129.6, 127.7, 127.3, 118.9, 110.6, 63.1, 61.3, 29.1, 26.3, 25.9, 16.3, 11.5; EIMS m/z (rel intensity) 464 (M⁺, 5.5), 466 (M⁺+2, 2.0), 397 (100), 299 (34), 98 (25); HRMS (EI) for C₂₅H₂₅³⁵ClN₄O₃ calcd 464.1615 (M⁺), found 464.1589, C₂₅H₂₅³⁷ClN₄O₃ calcd 466.1586 (M⁺+2), found 466.1551.

5-[(1Z)-1-(4-Cyanophenyl)-5-(2-oxo-1,3-oxazolidin-3-yl)pent-1-en-1-yl]-N,2-dimethoxy-3-methylbenzenecarboximidoyl Chloride (25). The general Stille coupling procedure was followed using 4-iodobenzennitrile 41 (58 mg, 0.25 mmol), organostannane 56 (202 mg, 0.303 mmol), CsF (115 mg, 0.759 mmol), Pd(PPh₃)₄ (29 mg, 0.025 mmol) and CuI (57 mg, 0.30 mmol) in dry DMF (10 mL). The reaction mixture was stirred for 20 h and purified by column chromatography on silica gel using 75% ethyl acetate-hexanes to give the product 25 (106 mg, 89.7%) as a clear oil: ¹H NMR (270 MHz, CDCl₃) δ 7.56 (d, J=6.7 Hz, 2 H), 7.32 (t, J=6.7 Hz, 2 H), 7.06 (s, 1 H), 7.01 (s, 1 H), 6.19 (t, J=7.4 Hz, 1 H), 4.26 (t, J=8.0 Hz, 2 H), 4.09 (s, 3 H), 3.85 (s, 3 H), 3.48 (t, J=8.0 Hz, 2 H), 3.27 (t, J=7.4 Hz, 2 H), 2.32 (s, 3 H), 2.23-2.14 (m, 2 H), 1.71 (quint, J=7.4 Hz, 2 H); ¹³C NMR (68 MHz, CDCl₃) δ 162.2, 158.1, 155.7, 146.1, 139.9, 134.3, 133.7, 132.4, 131.8, 131.7, 128.7, 127.5, 127.1, 118.7, 110.3, 62.9, 61.5, 61.2, 27.8, 27.2, 26.9, 16.3, 13.6; EIMS m/z (rel intensity) 467 (Mt, 12), 469 (Mt +2, 4.3), 400 (23), 313 (100), 287 (26), 100 (65), 56 (27); HRMS (EI) for C₂₅H₂₆³⁵ClN₃O₄ calcd 467.1612 (M⁺), found 467.1624, C₂₅H₂₆³⁷ClN₃O₄calcd 469.1582 (M⁺+2), found 469.1609.

tert-Butyl (5E)-6-(3,7-Dimethyl-2-oxo-2,3-dihydro-1,3-benzoxazol-5-yl)-6-{4-methoxy-3-methyl-5-[(methylsulfanyl)carbonyl]phenyl}hex-5-enoate (66). The general Stille coupling procedure was followed using aryl iodide 38 (405 mg, 1.41 mmol), organostannane 65 (1.10 g, 1.68 mmol), CsF (767 mg, 5.05 mmol), Pd(PPh₃)₄ (195 mg, 0.169 mmol) and CuI (38 mg, 0.202 mmol) in dry DMF (40 mL). The reaction mixture was stirred for 16 h and the product was purified by column chromatography on silica gel using 20% ethyl acetate-hexanes to give the product 66 (408 mg, 55.1%) as a colorless oil: ¹H NMR (270 MHz, CDCl₃) δ 7.38 (d, J=2.0 Hz, 1 H), 7.10 (d, J=2.0 Hz, 1 H), 6.75 (d, J=1.1 Hz, 1 H), 6.58 (d, J=1.1 Hz, 1 H), 5.96 (t, J=7.4 Hz, 1 H), 3.87 (s, 3 H), 3.35 (s, 3 H), 2.45 (s, 3 H), 2.41 (s, 6 H), 2.33-2.12 (m, 4 H), 1.81-1.73 (m, 2 H), 1.40 (s, 9 H); ¹³C NMR (68 MHz, CDCl₃) δ 191.9, 172.4, 155.3, 154.7, 140.3, 140.2, 138.6, 136.1, 135.1, 132.3, 131.1, 131.0, 129.8, 128.1, 123.4, 119.6, 104.5, 79.9, 61.6, 35.0, 29.2, 28.1, 28.0, 25.2, 16.1, 14.4, 12.4; EIMS m/z (rel intensity) 525 (M⁺, 2.2), 469 (36), 422 (100), 420 (32), 190 (15); HRMS (EI) for C₂₉H₃₅O₆NS calcd 525.2185 (M⁺), found 525.2150.

tert-Butyl (5Z)-6-(3-Methoxy-7-methyl-1,2-benzoxazol-5-yl)-6-{4-methoxy-3-methyl-5-[(methylsulfanyl)carbonyl]phenyl}hex-5-enoate (67). The general Stille coupling procedure was followed using aryl iodide 39 (405 mg, 1.41 mmol), organostannane 65 (1.10 g, 1.68 mmol), CsF (767 mg, 5.05 mmol), Pd(PPh₃)₄ (195 mg, 0.169 mmol) and CuI (39 mg, 0.20 mmol) in dry DMF (40 mL). The reaction mixture was stirred for 16 h and the product was purified by column chromatography on silica gel using 33% ethyl acetate-hexanes to give the product 67 (456 mg, 61.8%) as a colorless oil: ¹H NMR (270 MHz, CDCl₃) δ 7.41 (d, J=2.0 Hz, 1 H), 7.36 (d, J=2.0 Hz, 1 H), 7.08 (s, 2 H), 6.00 (t, J=7.4 Hz, 1 H), 3.87 (s, 3 H), 3.67 (s, 3 H), 2.44 (s, 3 H), 2.35 (s, 3 H), 2.32 (s, 3 H), 2.25-2.11 (m, 4 H), 1.79-1.68 (m, 2 H), 1.40 (s, 9 H); ¹³C NMR (68 MHz, CDCl₃) δ 191.5, 172.1, 162.6, 157.7, 155.3, 139.6, 138.2, 135.9, 134.7, 132.7, 132.3, 131.1, 129.9, 127.9, 119.6, 119.5, 115.4, 79.7, 61.4, 34.8, 32.4, 29.0, 27.8, 25.0, 16.0, 13.9, 12.2; EIMS m/z (rel intensity) 525 (M⁺, 0.22), 452 (18), 422 (100), 421 (28), 347 (17); HRMS (EI) for C₂₉H₃₅O₆NS calcd 525.2185 (M⁺), found 525.2171.

tert-Butyl (5Z)-6-(3-Cyanophenyl)-6-{4-methoxy-3-methyl-5-[methylsulfanyl)carbonyl]phenyl}hex-5-enoate (68). The general Stille coupling procedure was followed using 3-iodobenzonitrile 40 (321 mg, 1.40 mmol), organostannane 65 (1.10 g, 1.68 mmol), CsF (767 mg, 5.05 mmol), Pd(PPh₃)₄ (195 mg, 0.169 mmol) and CuI (39 mg, 0.20 mmol) in dry DMF (40 mL). The reaction mixture was stirred for 16 h and the product was the product was purified by column chromatography on silica gel using 15% ethyl acetate-hexanes to give the product 68 (679 mg, 99.9%) as a clear oil: ¹H NMR (270 MHz, CDCl₃) δ 7.52-7.35 (m, 4 H), 7.13 (s, 1 H), 7.08 (s, 1 H), 6.09 (t, J=7.4 Hz, 1 H), 3.88 (s, 3 H), 2.45 (s, 3 H), 2.34 (s, 3 H), 2.25-2.03 (m, 4 H), 1.75 (quint, J=7.4 Hz, 2 H), 1.36 (s, 9 H); ¹³C NMR (68 MHz, CDCl₃) δ 191.7, 172.5, 155.6, 143.1, 138.9, 136.2, 132.7, 132.0, 131.5, 131.4, 131.2, 130.5, 130.2, 128.8, 128.0, 118.6, 112.1, 80.0, 61.6, 34.9, 29.2, 28.0, 25.0, 16.1, 12.4; EIMS m/z (rel intensity) 465 (M⁺, 0.08), 409 (14), 362 (100), 259 (14).

tert-Butyl (5Z)-6-(4-Cyanophenyl)-6-{4-methoxy-3-methyl-5-[methylsulfanyl)carbonyl]phenyl}hex-5-enoate (69). The general Stille coupling procedure was followed using 4-iodobenzonitrile 41 (321 mg, 1.40 mmol), organostannane 65 (1.01 g, 1.68 mmol), CsF (767 mg, 5.05 mmol), Pd(PPh₃)₄ (195 mg, 0.169 mmol) and CuI (39 mg, 0.202 mmol) in dry DMF (40 mL). The reaction mixture was stirred for 16 h and purified by column chromatography on silica gel using 15% ethyl acetate-hexanes to give the product 69 (569 mg, 87.2%) as a clear oil: ¹H NMR (270 MHz, CDCl₃) δ 7.59 (d, J=8.6 Hz, 2 H), 7.34 (d, J=2.0 Hz, 1 H), 7.28 (d, J=8.6 Hz, 2 H), 7.08 (d, J=2.0 Hz, 1 H), 6.07 (t, J=7.4 Hz, 1 H), 3.86 (s, 3 H), 2.45 (s, 3 H), 2.33 (s, 3 H), 2.25-2.13 (m, 4 H), 1.75 (quint, J=7.4 Hz, 2 H), 1.39 (s, 9 H); ¹³C NMR (68 MHz, CDCl₃) δ 191.6, 172.2, 155.5, 146.2, 139.4, 136.0, 134.6, 133.8, 132.6, 131.7, 131.4, 128.0, 127.4, 118.6, 110.2, 79.9, 64.5, 34.9, 29.3, 28.0, 24.9, 16.1, 12.4; EIMS m/z (rel intensity) 465 (M⁺, 0.13), 109 (22), 363 (23), 362 (100).

S-Methyl 5-[(1E)-1-(3,7-Dimethyl-2-oxo-2,3-dihydro-1,3-benzoxazol-5-yl)-6-(methoxyamino)-6-oxohex-1-en-1-yl]-2-methoxy-3-methylbenzenecarbothioate (70). TFA (4 mL) was added to a solution of t-butyl ester 66 (428 mg, 0.814 mmol) in dry CH₂Cl₂ (4 mL) at 0° C. and the mixture was allowed to stir for 0.5 h at room temperature. The reaction mixture was concentrated under reduced pressure and the residue was dissolved with anhydrous CH₂Cl₂ (10 mL). The solution was added DIPEA (567 pt, 3.26 mmol), NH₂OCH₃.HCl (170 mg, 2.04 mmol), DMAP (49.7 mg, 0.407 mmol) and EDCI (390 mg, 2.03 mmol) at 0° C. and the mixture was stirred for 16 h at ambient temperature. The reaction was quenched with H₂O (10 mL) and extracted with CH₂Cl₂ (8 mL×3). The organic layers were dried over Na₂SO₄, filtered, and concentrated under reduced pressure. The residue was purified by silica gel column chromatography (hexanes-EtOAc-MeOH=50:50:3) to give the product 70 (266 mg, 65.5%) as a white amorphous solid: ¹H NMR (270 MHz, CDCl₃) δ 8.56 (s, 1 H), 7.37 (s, 1 H), 7.10 (s, 1 H), 6.74 (s, 1 H), 6.60 (s, 1 H), 5.96 (t, J=7.3 Hz, 1 H), 3.87 (s, 3 H), 3.71 (s, 3 H), 3.34 (s, 3 H), 2.45 (s, 3 H), 2.33 (s, 3 H), 2.32 (s, 3 H), 2.22-2.05 (m, 4 H), 1.83 (quint, J=7.3 Hz, 2 H); ¹³C NMR (68 MHz, CDCl₃) δ 192.0, 170.2, 155.4, 154.8, 140.4, 140.1, 138.5, 136.1, 135.0, 132.3, 131.0, 130.9, 129.6, 127.9, 123.4, 119.6, 104.5, 63.9, 61.5, 32.4, 29.2, 28.1, 25.4, 16.1, 14.3, 12.4; EIMS m/z (rel intensity) 498 (M⁺, 6.2), 451 (31), 419 (100), 403 (46), 321 (30), 190 (18); HRMS (EI) for C₂₆H₃₀O₆N₂S calcd 498.1825 (M⁺), found 498.1830.

S-Methyl 2-Methoxy-5-[(1Z)-6-(methoxyamino)-1-(3-methoxy-7-methyl-1,2-benzoxazol-5-yl)-6-oxohex-1-en-1-yl]-3-methylbenzenecarbothioate (71). Reaction and workup conditions were as described for compound 70. The crude product was purified by silica gel column chromatography (hexanes-EtOAc-MeOH=10:10:1) to give the product 71 (76.9%) as a white amorphous solid: ¹H NMR (270 MHz, CDCl₃) δ 9.36 (s, 1 H), 7.43 (s, 1 H), 7.35 (s, 1 H), 7.23 (s, 1 H), 7.01 (s, 1 H), 5.99 (t, J=7.4 Hz, 1 H), 3.87 (s, 3 H), 3.71 (s, 3 H), 3.66 (s, 3 H), 2.43 (s, 3 H), 2.34 (s, 3 H), 2.31 (s, 3 H), 2.22-2.05 (m, 4 H), 1.84 (quint, J=7.4 Hz, 2 H); ¹³C NMR (68 MHz, CDCl₃) δ 191.9, 170.3, 162.7, 157.7, 155.5, 139.8, 138.5, 136.1, 134.8, 133.1, 132.5, 131.1, 130.0, 128.0, 119.7, 119.6, 115.5, 63.9, 61.5, 32.5, 32.3, 29.1, 25.4, 16.1, 14.1, 12.3; EIMS m/z (rel intensity) 498 (M⁺, 6.4), 451 (53), 419 (100), 404 (37), 362 (19); HRMS (EI) for C₂₆H₃₀O₆N₂S calcd 498.1825 (M⁺), found 498.1849.

S-Methyl 5-[(1Z)-1-(3-Cyanophenyl)-6-(methoxyamino)-6-oxohex-1-en-1-yl]-2-methoxy-3-methylbenzenecarbothioate (72). Reaction and workup conditions were as described for compound 70. The crude product was purified by silica gel column chromatography (hexanes-EtOAc-MeOH=100:100:3) to give the product 72 (43.8%) as a white amorphous solid: ¹H NMR (270 MHz, CDCl₃) δ 8.53 (s, 1 H), 7.53-7.37 (m, 4 H), 7.35 (d, J=1.8 Hz, 1 H), 7.07 (d, J=1.8 Hz, 1 H), 6.10 (t, J=7.4 Hz, 1 H), 3.88 (s, 3 H), 3.70 (s, 3 H), 2.45 (s, 3 H), 2.34 (s, 3 H), 2.22-2.05 (m, 4 H), 1.83 (quint, J=7.4 Hz, 2 H); ¹³C NMR (68 MHz, CDCl₃) δ 191.9, 170.1, 155.7, 143.0, 139.0, 136.0, 133.8, 132.7, 131.6, 131.2, 131.1, 130.5, 130.2, 128.8, 127.9, 118.6, 112.0, 63.9, 61.5, 32.3, 29.2, 25.2, 16.1, 12.4; EIMS m/z (rel intensity) 438 (M⁺, 5.9), 391 (80), 359 (100), 344 (58), 316 (26), 302 (25); HRMS (EI) for C₂₄H₂₆O₄N₂S calcd 438.1614 (M⁺), found 438.1620.

S-Methyl 5-[(1Z)-1-(4-Cyanophenyl)-6-(methoxyamino)-6-oxohex-1-en-1-yl]-2-methoxy-3-methylbenzenecarbothioate (73). Reaction and workup conditions were as described for compound 70. The crude product was purified by silica gel column chromatography (hexanes-EtOAc-MeOH=25:25:1) to give the product 73 (46.3%) as a white amorphous solid: ¹H NMR (270 MHz, CDCl₃) δ 8.65 (s, 1 H), 7.55 (d, J=8.6 Hz, 2 H), 7.34 (d, J=1.6 Hz, 1 H), 7.28 (d, J=8.6 Hz, 2 H), 7.07 (d, J=1.6 Hz, 1 H), 6.17 (t, J=7.4 Hz, 1 H), 3.87 (s, 3 H), 3.69 (s, 3 H), 2.45 (s, 3 H), 2.33 (s, 3 H), 2.22-2.05 (m, 4 H), 1.83 (quint, J=7.4 Hz, 2 H); ¹³C NMR (68 MHz, CDCl₃) δ 191.9, 170.1, 155.6, 146.1, 139.5, 136.0, 133.8, 132.7, 132.5, 131.7, 131.2, 127.9, 127.4, 118.6, 110.0, 63.9, 61.5, 32.3, 29.2, 25.1, 16.0, 12.4; EIMS m/z (rel intensity) 438 (M⁺, 4.2), 391 (62), 359 (100), 343 (70), 316 (25), 273 (24); HRMS (EI) for C₂₄H2604N2S calcd 438.1614 (M⁺), found 438.1616.

S-Methyl 5-[(1E,6Z)-6-Chloro-1-(3,7-dimethyl-2-oxo-2,3-dihydro-1,3-benzoxazol-5-yl)-6-(methoxyimino)hex-1-en-1-yl]-2-methoxy-3-methylbenzenecarbothioate (26). CCl₄ (584 μL, 6.04 mmol) was added to a solution of methoxyamide 70 (251 mg, 0.503 mmol) and PPh₃ (396 mg, 1.51 mmol) in CH₃CN (18 mL) at room temperature and the reaction mixture was allowed to stir for 0.5 h at same temperature. The reaction mixture was heated at reflux temperature and stirred for 3 h. The mixture was concentrated under reduced pressure and the residue was purified by silica gel column chromatography using 20% ethyl acetate-hexanes to give the product 26 (233 mg, 89.5%) as a colorless oil: ¹H NMR (270 MHz, CDCl₃) δ 7.38 (d, J=2.2 Hz, 1 H), 7.09 (d, J=2.2 Hz, 1 H), 6.76 (s, 1 H), 6.59 (s, 1 H), 5.97 (t, J=7.4 Hz, 1 H), 3.90 (s, 3 H), 3.88 (s, 3 H), 3.35 (s, 3 H), 2.50 (t, J=7.4 Hz, 2 H), 2.45 (s, 3 H), 2.33 (s, 6 H), 2.23-2.15 (m, 2 H), 1.82 (quint, J=7.4 Hz, 2 H); ¹³C NMR (68 MHz, CDCl₃) δ 191.9, 155.4, 154.7, 140.7, 140.3, 138.5, 138.4, 136.0, 135.0, 132.4, 131.1, 129.3, 128.0, 123.4, 119.7, 104.5, 62.3, 61.6, 36.2, 28.7, 28.2, 26.4, 16.1, 14.4, 12.4; EIMS m/z (rel intensity) 516 (M⁺, 11), 518 (M⁺+2, 4.4), 471 (11), 469 (31), 401 (100); HRMS (EI) for C₂₆H₂₉³⁵ClN₂O₅S calcd 516.1486 (M⁺), found 516.1490, C₂₆H₂₉³⁷ClN₂O₅S calcd 518.1456 (M⁺), found 518.1455.

S-Methyl 5-[(1Z,6Z)-6-Chloro-6-(methoxyimino)-1-(3-methoxy-7-methyl-1,2-benzoxazol-5-yl)hex-1-en-1-yl]-2-methoxy-3-methylbenzenecarbothioate (27). Reaction and workup conditions were as described for compound 26. The crude product was purified by silica gel column chromatography using 25% ethyl acetate-hexanes to give the product 27 (92.5%) as a colorless oil: ¹H NMR (270 MHz, CDCl₃) δ 7.41 (s, 1 H), 7.36 (d, J=2.0 Hz, 1 H), 7.27 (s, 1 H), 7.07 (d, J=2.0 Hz, 1 H), 6.01 (t, J=7.4 Hz, 1 H), 3.90 (s, 3 H), 3.88 (s, 3 H), 3.67 (s, 3 H), 2.49 (t, J=7.4 Hz, 2 H), 2.44 (s, 3 H), 2.35 (s, 3 H), 2.32 (s, 3 H), 2.22-2.14 (m, 2 H), 1.80 (quint, J=7.4 Hz, 2 H); ¹³C NMR (68 MHz, CDCl₃) δ 191.8, 162.8, 157.9, 155.5, 140.1, 138.5, 138.3, 136.0, 134.8, 132.9, 132.5, 131.3, 129.5, 128.1, 119.8, 119.7, 115.7, 62.3, 61.6, 36.1, 32.6, 28.7, 26.4, 16.2, 14.1, 12.4; EIMS m/z (rel intensity) 516 (M⁺, 5.3), 518 (M⁺+2, 2.6), 471 (35), 469 (100), 403 (89), 401 (74), 362 (41); HRMS (EI) for C₂₆H₂₉³⁵ClN₂O₅S calcd 516.1486 (M⁺), found 516.1457, C₂₆H₂₉³⁷ClN₂O₅S calcd 518.1456 (M⁺+2), found 518.1498.

S-Methyl 5-[(1Z,6Z)-6-Chloro-1-(3-cyanophenyl)-6-(methoxyimino)hex-1-en-1-yl]-2-methoxy-3-methylbenzenecarbothioate (28). Reaction and workup conditions were as described for compound 26. The crude product was purified by silica gel column chromatography using 15% ethyl acetate-hexanes to give the product 28 (84.4%) as a colorless oil: ¹H NMR (270 MHz, CDCl₃) δ 7.54-7.38 (m, 4 H), 7.35 (d, J=2.0 Hz, 1 H), 7.07 (d, J=2.0 Hz, 1 H), 6.10 (t, J=7.4 Hz, 1 H), 3.89 (s, 3 H), 3.89 (s, 3 H), 2.49 (t, J=7.4 Hz, 2 H), 2.45 (s, 3 H), 2.34 (s, 3 H), 2.24-2.16 (m, 2 H), 1.82 (quint, J=7.4 Hz, 2 H); ¹³C NMR (68 MHz, CDCl₃) δ 191.7, 155.7, 143.0, 139.3, 138.4, 135.9, 133.8, 132.8, 131.4, 131.3, 131.2, 130.6, 130.3, 128.8, 128.0, 118.6, 112.2, 62.3, 61.7, 36.1, 28.7, 26.2, 16.2, 12.5; EIMS m/z (rel intensity) 456 (M⁺, 5.2), 458 (M⁺+2, 2.1), 411 (34), 409 (100), 343 (79), 341 (57); HRMS (EI) for C₂₄H25³⁵ClN203S calcd 456.1274 (M⁺), found 456.1243, C₂₆H₂₉³⁷ClN₂O₅calcd 458.1245 (M⁺+2), found 458.1221.

S-Methyl 5-[(1Z,6Z)-6-Chloro-1-(4-cyanophenyl)-6-(methoxyimino)hex-1-en-1-yl]-2-methoxy-3-methylbenzenecarbothioate (29). Reaction and workup conditions were as described for compound 26. The crude product was purified by silica gel column chromatography using 15% ethyl acetate-hexanes to give the product 29 (85.3%) as a colorless oil: ¹H NMR (270 MHz, CDCl₃) δ 7.56 (d, J=8.8 Hz, 2 H), 7.35 (d, J=2.2 Hz, 1 H), 7.29 (d, J=8.8 Hz, 2 H), 7.07 (d, J=2.2 Hz, 1 H), 6.17 (t, J=7.4 Hz, 1 H), 3.89 (s, 3 H), 3.87 (s, 3 H), 2.49 (t, J=7.4 Hz, 2 H), 2.45 (s, 3 H), 2.33 (s, 3 H), 2.25-2.16 (m, 2 H), 1.82 (quint, J=7.4 Hz, 2 H); ¹³C NMR (68 MHz, CDCl₃) δ 191.8, 155.7, 146.2, 139.9, 138.4, 135.9, 133.8, 132.8, 132.2, 131.8, 131.5, 128.0, 127.5, 118.7, 110.4, 62.4, 61.7, 36.1, 28.8, 26.2, 16.2, 12.5; EIMS m/z (rel intensity) 456 (M⁺, 7.1), 458 (M⁺+2, 2.7), 411 (34), 409 (100), 373 (44), 343 (73), 341 (62); HRMS (EI) for C₂₄H25³⁵ClN203S calcd 456.1274 (M⁺), found 456.1259, C₂₆H₂₉³⁷ClN₂O₅calcd 458.1245 (M⁺+2), found 458.1228.

Those skilled in the art will recognize that numerous modifications can be made to the specific implementations described above. The implementations should not be limited to the particular limitations described. Other implementations may be possible.

It is intended that that the scope of the present methods and compositions be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

1. A compound of formula (I) 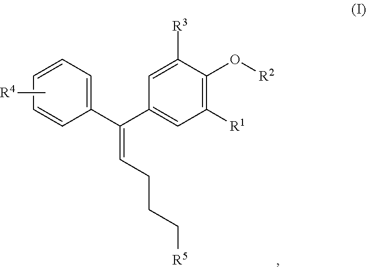
or a pharmaceutically acceptable salt, hydrate, prodrug, polymorph, or solvate thereof, wherein
R¹ is hydrogen, an alkyl, alkenyl, alkynyl, heteroalkyl, or heteroalkenyl;
R² is an alkyl or acyl;
R³ is an acyl or an imidoyl;
R⁴ represents five substituents each independently selected from the group consisting of hydrogen, halo, and cyano; and
R⁵ is an acyl, alkyl, alkenyl, heteroalkyl, heteroalkenyl, heterocyclyl, cycloalkyl, cycloalkenyl, cycloheteroalkyl, cycloheteroalkenyl, aryl, arylalkyl, or arylalkenyl, each of which is optionally substituted.

2. The compound according to claim 1, wherein R¹ is a C₁-C₆ alkyl.

3. The compound according to claim 1, wherein R² is a C₁-C₆ alkyl.

4. The compound according to claim 1, wherein R³ is an N-alkoxyimidoyl halide.

5. The compound according to claim 1, wherein R³ is an acyl thioester and R⁵ is an N-alkoxyimidoyl halide.

6. The compound according to claim 1, wherein one of said five R⁴ substituents is cyano (—CN) and other four are hydrogen.

7. The compound according to claim 1, wherein R⁴ represents five substituents wherein two adjacent substituents are taken together with the attached carbons/heteroatoms to form an optionally substituted cyclic or heterocyclic moiety.

8. The compound according to claim 1, wherein R⁵ is an N-alkoxyimidoyl halide.

9. The compound according to claim 1, wherein the compound is selected from the group consisting of

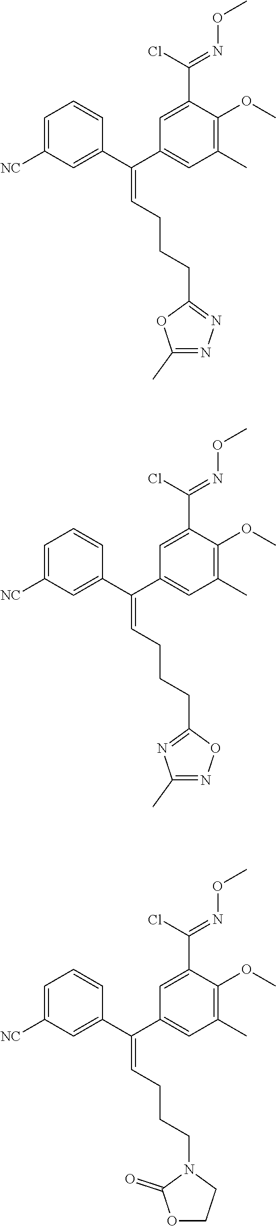 [SMILES: CO/N=C(\Cl)c1cc(/C(=C\CCCc2nnc(C)o2)c2cccc(C#N)c2)cc(C)c1OC ; CO/N=C(\Cl)c1cc(/C(=C\CCCc2nc(C)no2)c2cccc(C#N)c2)cc(C)c1OC ; CO/N=C(\Cl)c1cc(/C(=C\CCCN2CCOC2=O)c2cccc(C#N)c2)cc(C)c1OC]

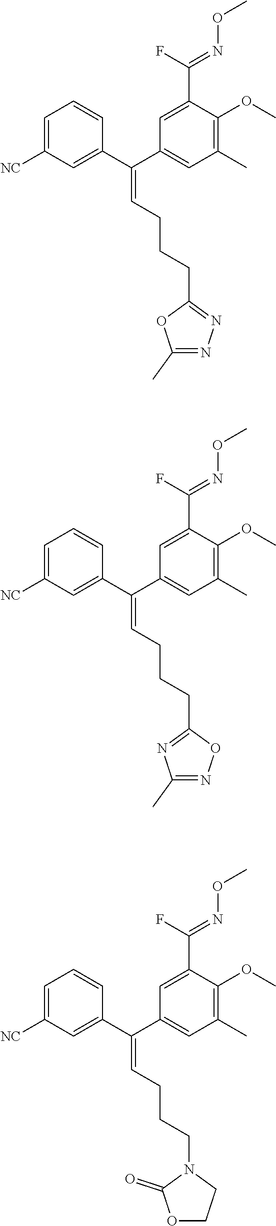 [SMILES: CO/N=C(\F)c1cc(/C(=C\CCCc2nnc(C)o2)c2cccc(C#N)c2)cc(C)c1OC ; CO/N=C(\F)c1cc(/C(=C\CCCc2nc(C)no2)c2cccc(C#N)c2)cc(C)c1OC ; CO/N=C(\F)c1cc(/C(=C\CCCN2CCOC2=O)c2cccc(C#N)c2)cc(C)c1OC]

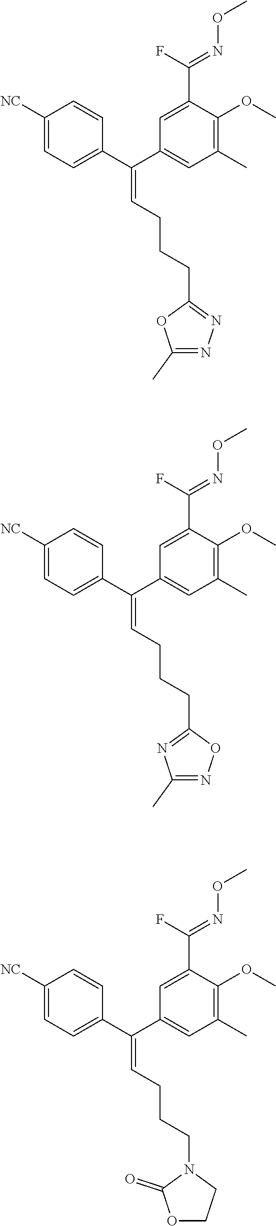 [SMILES: CO/N=C(\F)c1cc(/C(=C\CCCc2nnc(C)o2)c2ccc(C#N)cc2)cc(C)c1OC ; CO/N=C(\F)c1cc(/C(=C\CCCc2nc(C)no2)c2ccc(C#N)cc2)cc(C)c1OC ; CO/N=C(\F)c1cc(/C(=C\CCCN2CCOC2=O)c2ccc(C#N)cc2)cc(C)c1OC]

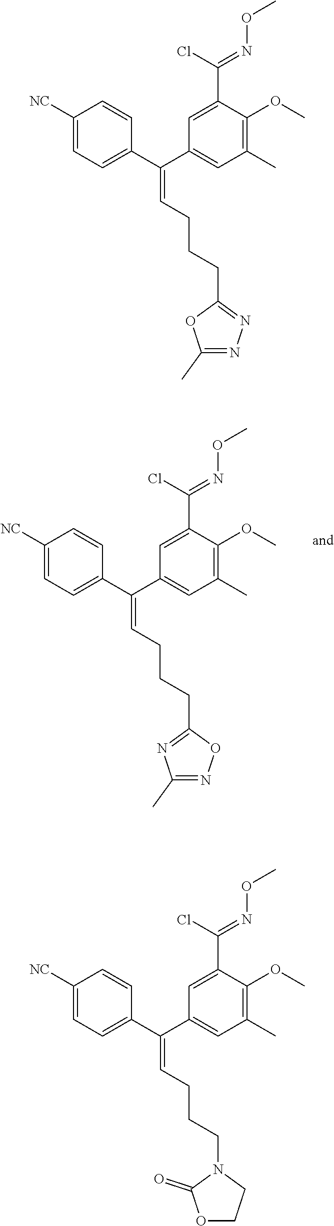 [SMILES: CO/N=C(\Cl)c1cc(/C(=C\CCCc2nnc(C)o2)c2ccc(C#N)cc2)cc(C)c1OC ; CO/N=C(\Cl)c1cc(/C(=C\CCCc2nc(C)no2)c2ccc(C#N)cc2)cc(C)c1OC ; CO/N=C(\Cl)c1cc(/C(=C\CCCN2CCOC2=O)c2ccc(C#N)cc2)cc(C)c1OC]

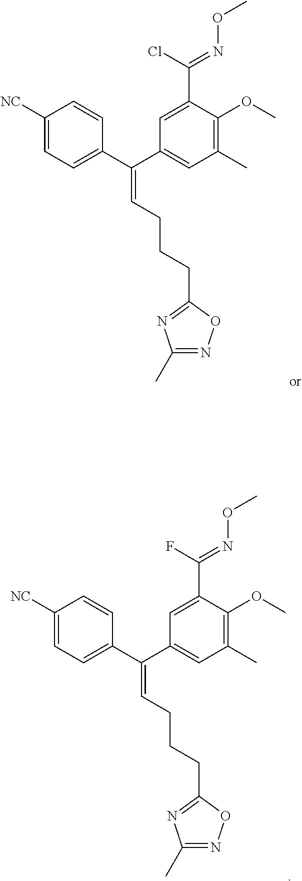 [SMILES: CO/N=C(\Cl)c1cc(/C(=C\CCCc2nc(C)no2)c2ccc(C#N)cc2)cc(C)c1OC ; CO/N=C(\F)c1cc(/C(=C\CCCc2nc(C)no2)c2ccc(C#N)cc2)cc(C)c1OC]

11. A pharmaceutical composition comprising a compound of claim 1, or a pharmaceutically acceptable salt thereof, together with one or more diluents, excipients or carriers.

12. A pharmaceutical composition comprising a compound of claim 1, or a pharmaceutically acceptable salt thereof, in combination with one or more other therapeutically active compounds by the same or different modes of action, together with one or more diluents, excipients or carriers.

13. A method of treatment of a patient with HIV infection comprising the step of administrating a therapeutically effective amount of a compound of claim 1, or a pharmaceutically acceptable salt thereof, to the patient in need of relief from said infection.

14. A method of treatment of a patient with HIV infection comprising the step of administrating a therapeutically effective amount of a compound of claim 1, or a pharmaceutically acceptable salt thereof, in combination with one or more other therapeutically active compounds of the same or different modes of action, to the patient in need of relief from said infection.

15. (Withdrawn, currently amended) A method for the treatment of a patient with HIV infection comprising administration to the patient in need of relief from said infection a therapeutically effective amount of a compound of formula (I) 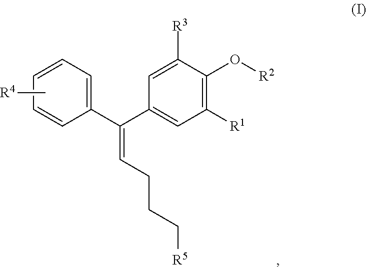
or a pharmaceutically acceptable salt, hydrate, prodrug, polymorph, or solvate thereof, wherein
R¹ is hydrogen, an alkyl, alkenyl, alkynyl, heteroalkyl, or heteroalkenyl;
R² is an alkyl or acyl;
R³ is an acyl or an imidoyl;
R⁴ represents five substituents each independently selected from the group consisting of hydrogen, halo, and cyano; and
R⁵ is acyl, alkyl, alkenyl, heteroalkyl, heteroalkenyl, heterocyclyl, cycloalkyl, cycloalkenyl, cycloheteroalkyl, cycloheteroalkenyl, aryl, arylalkyl, or arylalkenyl, each of which is optionally substituted.

16. The method for the treatment of a patient with HIV infection according to claim 15, wherein the method comprise the step of administrating a therapeutic effective amount of compound 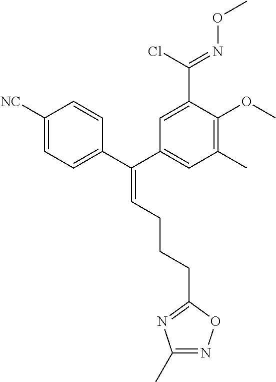 [SMILES: CO/N=C(\Cl)c1cc(/C(=C\CCCc2nc(C)no2)c2ccc(C#N)cc2)cc(C)c1OC]
or the pharmaceutically acceptable salt, hydrate, prodrug, polymorph, or solvate thereof, together with one or more carriers, excipients, or diluents, to the patient in need of relief from said infection.

17. The method for the treatment of a patient with HIV infection according to claim 15, wherein the method comprise the step of administrating a therapeutic effective amount of compound 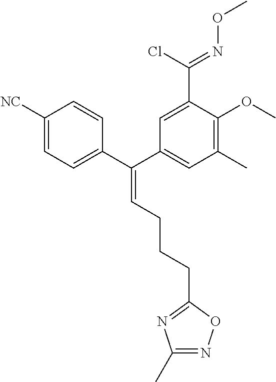 [SMILES: CO/N=C(\Cl)c1cc(/C(=C\CCCc2nc(C)no2)c2ccc(C#N)cc2)cc(C)c1OC]
or the pharmaceutically acceptable salt, hydrate, prodrug, polymorph, or solvate thereof, and one or more other therapeutically effective compounds of the same or different mode of action, together with one or more carriers, excipients, or diluents, to the patient in need of relief from said infection.

18. The method for the treatment of a patient with HIV infection according to claim 15, wherein the method comprise the step of administrating a therapeutic effective amount of compound 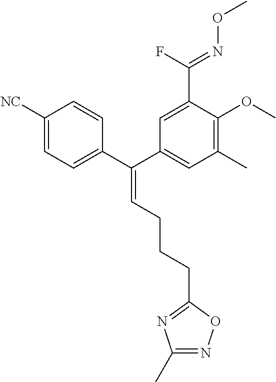 [SMILES: CO/N=C(\F)c1cc(/C(=C\CCCc2nc(C)no2)c2ccc(C#N)cc2)cc(C)c1OC]
or the pharmaceutically acceptable salt, hydrate, prodrug, polymorph, or solvate thereof, together with one or more carriers, excipients, or diluents, to the patient in need of relief from said infection.

19. The method for the treatment of a patient with HIV infection according to claim 15, wherein the method comprise the step of administrating a therapeutic effective amount of compound 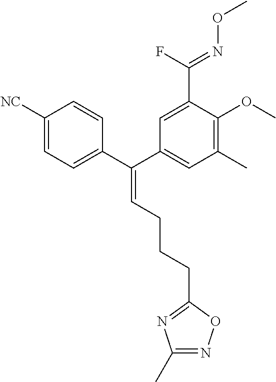 [SMILES: CO/N=C(\F)c1cc(/C(=C\CCCc2nc(C)no2)c2ccc(C#N)cc2)cc(C)c1OC]
or the pharmaceutically acceptable salt, hydrate, prodrug, polymorph, or solvate thereof, and one or more other therapeutically effective compounds of the same or different mode of action, together with one or more carriers, excipients, or diluents, to the patient in need of relief from said infection.
